SEQUENCE LISTING (3 sequences)

SEQ ID NO: 1 - protein, 959 aa, Streptococcus pyogenes
EEKTVQVQKGLPSIDSLHYLSENSKKEFKEELSKAGQESQKVKEILAKAQQADKQAQELAKMKIPEKIPMKPLHGPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

SEQ ID NO: 2 - protein, 995 aa, Streptococcus pyogenes
MDKHLLVKRTLGCVCAATLMGAALATHHDSLNTVKAEEKTVQVQKGLPSIDSLHYLSENSKKEFKEELSKAGQESQKVKEILAKAQQADKQAQELAKMKIPEKIPMKPLHGPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

SEQ ID NO: 3 - DNA, 3403 bp, Streptococcus pyogenes
ctcttttgtcctgccatggatggcaggttggcaaaaaaatgagaaaagcctaaaaaccttaaatttgtgttattttacctatactttgtaccttgttttttttaataaagtgttgttatacttaaggcgaactataggaatgcgcttacatggatggtatatcaactgggaagccatgacttagtaccaaaaataaggagtgtccaaatggataaacatttgttggtaaaaagaacactagggtgtgtttgtgctgcaacgttgatgggagctgccttagcgacccaccatgattcactcaatactgtaaaagcggaggagaagactgttcaggttcagaaaggattaccttctatcgatagcttgcattatctgtcagagaatagcaaaaaagaatttaaagaagaactctcaaaagcggggcaagaatctcaaaaggtcaaagagatattagcaaaagctcagcaggcagataaacaagctcaagaacttgccaaaatgaaaattcctgagaaaataccgatgaaaccgttacatggtcctctctacggtggttactttagaacttggcatgacaaaacatcagatccaacagaaaaagacaaagttaactcgatgggagagcttcctaaagaagtagatctagcctttattttccacgattggacaaaagattatagccttttttggaaagaattggccaccaaacatgtgccaaagttaaacaagcaagggacacgtgtcattcgtaccattccatggcgtttcctagctgggggtgataacagtggtattgcagaagataccagtaaatacccaaatacaccagagggaaataaagctttagccaaagctattgttgatgaatatgtttataaatacaaccttgatggcttagatgtggatgttgaacatgatagtattccaaaagttgacaaaaaagaagatacagcaggcgtagaacgctctattcaagtgtttgaagaaattgggaaattaattggaccaaaaggtgttgataaatcgcggttatttattatggatagcacctacatggctgataaaaacccattgattgagcgaggagctccttatattaatttattactggtacaggtctatggttcacaaggagagaaaggtggttgggagcctgtttctaatcgacctgaaaaaacaatggaagaacgatggcaaggttatagcaagtatattcgtcctgaacaatacatgattggtttttctttctatgaggaaaatgctcaagaagggaatctttggtatgatattaattctcgcaaggacgaggacaaagcaaatggaattaacactgacataactggaacgcgtgccgaacggtatgcaaggtggcaacctaagacaggtggggttaagggaggtatcttctcctacgctattgaccgagatggtgtagctcatcaacctaaaaaatatgctaaacagaaagagtttaaggacgcaactgataacatcttccactcagattatagtgtctccaaggcattaaagacagttatgctaaaagataagtcgtatgatctgattgatgagaaagatttcccagataaggctttgcgagaagctgtgatggcgcaggttggaaccagaaaaggtgatttggaacgtttcaatggcacattacgattggataatccagcgattcaaagtttagaaggtctaaataaatttaaaaaattagctcaattagacttgattggcttatctcgcattacaaagctcgaccgttctgttttacccgctaatatgaagccaggcaaagataccttggaaacagttcttgaaacctataaaaaggataacaaagaagaacctgctactatcccaccagtatctttgaaggtttctggtttaactggtctgaaagaattagatttgtcaggttttgaccgtgaaaccttggctggtcttgatgccgctactctaacgtctttagaaaaagttgatatttctggcaacaaacttgatttggctccaggaacagaaaatcgacaaatttttgatactatgctatcaactatcagcaatcatgttggaagcaatgaacaaacagtgaaatttgacaagcaaaaaccaactgggcattacccagatacctatgggaaaactagtctgcgcttaccagtggcaaatgaaaaagttgatttgcaaagccagcttttgtttgggactgtgacaaatcaaggaaccctaatcaatagcgaagcagactataaggcttaccaaaatcataaaattgctggacgtagctttgttgattcaaactatcattacaataactttaaagtttcttatgagaactataccgttaaagtaactgattccacattgggaaccactactgacaaaacgctagcaactgataaagaagagacctataaggttgacttctttagcccagcagataagacaaaagctgttcatactgctaaagtgattgttggtgacgaaaaaaccatgatggttaatttggcagaaggcgcaacagttattggaggaagtgctgatcctgtaaatgcaagaaaggtatttgatgggcaactgggcagtgagactgataatatctctttaggatgggattctaagcaaagtattatatttaaattgaaagaagatggattaataaagcattggcgtttcttcaatgattcagcccgaaatcctgagacaaccaataaacctattcaggaagcaagtctacaaatttttaatatcaaagattataatctagataatttgttggaaaatcccaataaatttgatgatgaaaaatattggattactgtagatacttacagtgcacaaggagagagagctactgcattcagtaatacattaaataatattactagtaaatattggcgagttgtctttgatactaaaggagatagatatagttcgccagtagtccctgaactccaaattttaggttatccgttacctaacgccgacactatcatgaaaacagtaactactgctaaagagttatctcaacaaaaagataagttttctcaaaagatgcttgatgagttaaaaataaaagagatggctttagaaacttctttgaacagtaagatttttgatgtaactgctattaatgctaatgctggagttttgaaagattgtattgagaaaaggcagctgctaaaaaaataaacaaagtaactttcttagatagcaacattcagattaaattaacaaaatgtgactatgataaaggtttgctggaattgattaaccaaaagactaaaaatctgagatgaatagtcccagatttttagtcttttataggttttgatgacataaagctaaataatcgttagactaccagaaagggcgcttgtccgtgagacatggctgtctt

(19) United States
(12) Patent Application Publication
(10) Pub. No.: US 2011/0318340 A1
(43) Pub. Date: Dec. 29, 2011

(54) DEGLYCOSYLATED ANTIBODIES

(75) Inventors: Mattias Collin, Lund (SE); Maria Allhorn, Ramlosa (SE); Rikard Holmdahl, Stockholm (SE); Kutty Selva Nandakumar, Uppsala (SE)

(73) Assignee: Hansa Medical AB, Lund (SE)

(21) Appl. No.: 13/129,788

(22) PCT Filed: Nov. 18, 2009
(86) PCT No.: PCT/EP2009/008215

§ 371 Date: Sep. 9, 2011

(30) Foreign Application Priority Data
Nov. 18, 2008 (GB) 0821100.5

(51) Int. Cl. A61K 39/395; C07K 16/00; C07K 16/34; C07H 21/00; A61P 37/06; A61P 29/00; A61P 17/06; A61P 19/02; A61P 17/00; A61P 3/10; A61P 9/00; A61P 13/12; A61P 7/06; A61P 1/00; A61P 7/00; A61P 25/28; A61P 27/16; A61P 43/00; A61P 1/16; A61P 35/00; C07K 16/18

(52) U.S. Cl. 424/133.1; 530/387.3; 536/23.53

(57) ABSTRACT

The present invention provides an antibody, or antibody fragment, with modified glycosylation for use in treating or preventing a disease or condition, where the antibody or fragment: (a) suppresses an inflammatory condition forming part of the disease or condition to be treated or prevented; and/or (b) displays increased efficacy and/or decreased side effects in comparision to treatment or prevention with the corresponding antibody with unmodified glycosylation.

### FIELD OF THE INVENTION

The present invention relates to antibodies, and antibody fragments, which have modified glycosylation and their use in treatment. Particularly, the invention relates to using such antibodies and fragments to suppress an inflammatory condition.

### BACKGROUND OF THE INVENTION

A large number of diseases and conditions involve unwanted inflammatory conditions. One illustration of a condition involving a damaging inflammatory condition is Rheumatoid Arthritis (RA). RA is a crippling and widespread disease, involving joint inflammation and damage. A variety of antibodies against self-antigens are found in RA sufferers, including antibodies against Type II Collagen. It is believed that such antibodies play an important role in the condition, with administration of monoclonal antibodies against Type II collagen being able to induce arthritis in a mouse model of the disease.

There is a need for ways to treat conditions such as RA by reducing or eliminating inflammatory condition underlying the disorders.

### SUMMARY OF THE INVENTION

The present invention is based on the finding that antibodies, or antibody fragments, having modified glycosylation may be used therapeutically. Surprisingly the antibodies and fragments may be used to suppress inflammatory conditions. Furthermore, the modification of glycosylation may be used to increase the efficacy of existing antibody-based treatments. The antibodies and fragments of the invention display increased potency and decreased side effects in comparison to their normally glycosylated counterparts. The invention therefore represents a considerable advance in treating inflammatory conditions.

Whilst not wanting to be constrained to a particular theory, it is believed that at least part of the invention may reside in how the antibody or fragment interacts with the immune system and in particular suppresses the immune system. Thus the effect may be used both to treat an inflammatory condition, but also to increase potency and to reduce side effects of therapeutic antibodies through this modification of interaction with the immune system.

Thus, the present invention provides an antibody, or antibody fragment, with modified glycosylation for use in treating or preventing a disease or condition, where the encoded antibody or fragment: (a) suppresses an inflammatory condition forming part of the disease or condition to be treated or prevented; (b) and/or displays increased efficacy and/or decreased side effects in comparision to treatment or prevention with the corresponding antibody with unmodified glycosylation.

The invention also provides for a polynucleotide encoding an antibody, or antibody fragment, with modified glycosylation for use in treating or preventing a disease or condition, where the antibody or fragment: (a) suppresses an inflammatory condition forming part of the disease or condition to be treated or prevented; (b) and/or displays increased efficacy and/or decreased side effects in comparision to treatment or prevention with the corresponding antibody with unmodified glycosylation.

Additionally, the invention provides for use of an antibody, or antibody fragment, with modified glycosylation in the manufacture of a medicament for treating or preventing a disease or condition, where the antibody or fragment: (a) suppresses an inflammatory condition forming part of the disease or condition to be treated or prevented; (b) and/or displays increased efficacy and/or decreased side effects in comparision to treatment or prevention with the corresponding antibody with unmodified glycosylation.

The invention further provides a method of treating or preventing a disease or condition in a subject in need comprising administering to the subject a therapeutically effective amount of an antibody, or antibody fragment, with modified glycosylation, where the antibody or fragment: (a) suppresses an inflammatory condition forming part of the disease or condition to be treated or prevented; (b) and/or displays increased efficacy and/or decreased side effects in comparision to treatment or prevention with the corresponding antibody with unmodified glycosylation.

### BRIEF DESCRIPTION OF FIGURES

FIG. 1, panels (A) and (B) shows how anti-CII antibodies with modified glycosylation dominantly suppress induction of CAIA (Collagen Antigen Induced Arthritis). Mice were administered: (i) fully glycosylated monoclonal antibodies; (ii) the same monoclonal antibodies, but with modified glycosylation; or (iii) mixtures of both at differing ratios of glycosylated to modified forms. Panel (A) shows arthritis incidence and panel (B) mean arthritis score of arthritic animals only, with n indicating the number of mice used in each group.

FIG. 1, panels (C) and (D) shows that antibodies with modified glycosylation are capable of dominantly suppressing induction of arthritis by monoclonal antibodies recognising different epitopes to the antibodies with modified glycosylation. The closed circles show the results for a mixture of four different monoclonal antibodies in glycosylated form, with the open circles showing the results for a mixture where two of the four antibodies have modified glycosylation. Panel (C) shows arthritis incidence and panel (D) mean arthritis score of arthritic animals only, with n− indicating the number of mice used in each group.

FIG. 2 shows hydrolysis of human anti-D IgG by EndoS. Panel (A) shows quantification of RBC-bound IgG by solubilisation of sensitized RBC followed by Western blot using antiserum against human IgG. Panel (B) shows quantification of the amount of RBC-bound IgG by ELISA using antiserum against human IgG. Panel (C) shows glycan hydrolysis of RBC-bound IgG determined by lectin ELISA. For each panel the presence or absence of anti-D and EndoS in the test sample is indicated at the bottom.

FIG. 3 shows that red blood cells sensitised with anti-D IgG pre-treated with EndoS are not phagocytosed by monocytes. Panel (A) shows the degree of adherence and phagocytosis determined by monocyte monolayer assay (MMA) and expressed as the number monocytes with one or more adherent/phagocytosed RBC out of 100 monocytes detected. Panel (B) shows the appearance of “rosettes” showed using Grünvald-Giemsa staining of monocytes monolayers incubated with RBC sensitized with anti-D at the top and below the results obtained when the anti-D was pre-treated with EndoS.

FIG. 4 shows intracellular ingestion of anti-D sensitized RBC and accumulation of haemoglobin is inhibited by pre-treatment of anti-D with EndoS. Panel (A) shows ingestion of sensitized RBC by THP-1 cells and blood monocytes presented as the amount of internalized haemoglobin detected by reactivity with 2,7-diaminofluorene. Panel (B) shows the degree of ingested RBC determined by measuring the fluorescence as measured via purified blood monocytes being incubated with FITC-labelled sensitized RBC. Panel (C) shows resuspended monocytes after incubation with anti-D sensitized RBC followed by the removal of uningested RBC, with the left photo showing RBC sensitized with anti-D and the right, anti-D pre-treated with EndoS.

FIG. 5 shows hydrolysis of anti-human RBC-IgG by EndoS. Panel (A) shows Western blot results for RBC bound IgG, where rabbit antisera against human RBC treated with EndoS or PBS was used for sensitization of RBC and bound IgG detected using antiserum against rabbit IgG. Panel (B) shows the amount of RBC-bound antibody as quantified by ELISA using antiserum against rabbit IgG. Panel (C) shows glycan hydrolysis of RBC-bound IgG as determined by lectin ELISA.

FIG. 6 shows EndoS inhibits hemolysis and agglutination of RBC sensitized with anti-RBC IgG. Panel (A) shows haemoglobin content quantified using 2,7-diaminofluorene staining. Rabbit IgG was directed against human RBC, pre-treated with EndoS or not and added to whole blood and after one hour incubation the blood cells were pelleted and supernatants analyzed for haemoglobin. For (1), instead of the pre-treatment of antibody, EndoS was directly added to blood and incubated one hour. The relative amount of haemoglobin is presented as a mean of four experiments and standard error of the mean. Panel (B) shows C1q deposition into sensitized RBC detected using flourescein-conjugated anti-human C1q. Anti-RBC IgG, 80 μg, was added to 700 μl human blood, diluted 35 times in PBS and incubated 1 hour at 37° C.

FIG. 7, panel (A) shows EndoS inhibiting binding of C1q to IgG-sensitized RBC and secretion of IL-8 by monocytes induced by sensitized RBC. Human serum was added to RBC freshly sensitized with rabbit antisera against human RBC, with or without pretreatment of antibody with EndoS. C1q deposition into sensitized RBC was detected using flourescein-conjugated anti-human C1q. Panel (B) shows IL-8 content. THP-1 cells or monocytes were purified from human blood were incubated with sensitized RBC, with or without pre-treatment of anti-D with EndoS, for 3 hours and the supernatants analyzed for IL-8.

FIG. 8 shows treatment of RBC sensitized with human immune plasma with EndoS inhibits oxygen metabolite production by monocytes exposed to sensitized RBC. RBC were sensitized with DAT positive human plasma from three patients and subsequently treated with EndoS before addition to purified blood monocytes. The secretion of oxygen metabolites from monocytes was detected by measuring the chemiluminescence.

FIG. 9 panels (A) and (B) shows that antibodies with modified glycosylation are capable of dominantly suppressing induction of arthritis by monoclonal antibodies recognising overlapping or non-overlapping epitopes to the antibodies with modified glycosylation. The triangles show the results for mice treated with a pair of antibodies having modified glycosylation which recognise epitopes that do not overlap with the epitopes recognised by the arthritogenic antibodies. The squares show the results for mice treated with a pair of antibodies having modified glycosylation which recognise epitopes that do overlap with the epitopes recognised by the arthritogenic antibodies. The circles show the results for mice which were not treated with antibodies having modified glycosylation. Panel (A) shows arthritis incidence and panel (B) mean arthritis score of arthritic animals only. n=11 for the non-overlapping epitope group and the no treatment group. n=5 for the overlapping epitope group.

FIG. 10 panels (A) and (B) shows that antibodies with modified glycosylation are capable of dominantly suppressing induction of arthritis in animals with and without a spleen. The open diamonds show animals which underwent splenectomy and were treated with a pair of antibodies having modified glycosylation. The open circles show animals which underwent sham splenectomy and were treated with a pair of antibodies having modified glycosylation. The closed diamonds show animals which underwent splenectomy and were untreated. The closed circles show animals which underwent sham splenectomy and were untreated. Panel (A) shows arthritis incidence and panel (B) mean arthritis score of arthritic animals only. n indicates the number of animals in each group.

FIG. 11 panels (A) and (B) shows that antibodies with modified glycosylation are capable of dominantly suppressing arthritis independent of antigen specificity. The closed diamonds show animals which were treated with a pair of antibodies having modified glycosylation, said antibodies being specific for different antigens to the disease-inducing antibodies. The closed circles show animals which were untreated. Panel (A) shows arthritis incidence and panel (B) mean arthritis score of arthritic animals only. n indicates the number of animals in each group.

FIG. 12 panels (A) and (B) shows that a single antibody with modified glycosylation is capable of dominantly suppressing arthritis at even low dose. The closed diamonds show animals which were treated with a single antibody having modified glycosylation (M2139D) at 50 μg, the open squares show animals which were treated with M2139D at 250 μg, the open circles show animals which were treated with M2139D at 1000 μg, and the open triangles show animals which were treated with M2139D at 4000 μg. The closed circles show animals which were untreated. Panel (A) shows arthritis incidence and panel (B) mean arthritis score of arthritic animals only. n indicates the number of animals in each group.

FIG. 13 shows a schematic representation of the glycosyl chains present at Asn-297 of the heavy chains of naturally occurring IgG molecules. This population of glycan chains contains three sets of glycoforms termed IgG-G0, -G1 and -G2. The IgG-G2 (FIG. 13A) biantennary glycans at Asn-297 have two arms that terminate in galactose residues. Approximately 16% of total IgG glycosyl chains take this form. The IgG-G1 (FIG. 13B) glycans lack a terminal galactose residue on one biantennary arm. Approximately 35% of total IgG glycosyl chains take this form. The IgG-G0 (FIG. 13C) glycans lack a terminal galactose residue on both biantennary arms. Approximately 35% of total IgG glycosyl chains take this form. Another 14% of total IgG glycosyl chains consists of IgG-G1 or IgG-G2 forms which are sialylated. Further variety is introduced, in that some glycans also have a bisecting GlcNAc residue connected to the first mannose residue (approximately 30% of the total IgG1 glycan pool), a core fucose attached to the first core GlcNAc residue (approximately 70% of the total IgG1 glycan pool), and sialylation of the terminal 1,3 arm galactose residues (approximately 14% of the total IgG1 glycan pool). The dashed line indicates the position at which EndoS cleaves these structures.

### BRIEF DESCRIPTION OF THE SEQUENCES

SEQ ID NO: 1 is an amino acid sequence of EndoS from S. pyogenes AP1.

SEQ ID NO: 2 is an amino acid sequence of EndoS from S. pyogenes AP1, including a signal sequence.

SEQ ID NO: 3 is a nucleic acid sequence encoding EndoS from S. pyogenes AP1, including a signal sequence.

### DETAILED DESCRIPTION OF THE INVENTION

The present invention is concerned with the use of antibodies, or antibody fragments, with modified glycosylation. The antibodies and fragments display the ability to suppress an inflammatory condition forming part of the disease or condition to be treated or prevented. In some instances, the disease or condition may comprise, consist of, or consist essentially of, the inflammatory condition.

Furthermore, where corresponding antibodies, or antibody fragments, with normal glycosylation have been previously employed, those of the invention with modified glycosylation display increased potency and/or reduced side-effects.

The present invention thus provides an antibody, or antibody fragment, with modified glycosylation for use in treating or preventing a disease or condition, where the antibody or fragment: (a) suppresses an inflammatory condition forming part of the disease or condition to be treated or prevented; and/or (b) displays increased efficacy and/or decreased side effects in comparision to treatment or prevention with the corresponding antibody with unmodified glycosylation.

The antibody or antibody fragment of the invention may therefore be used to suppress inflammatory conditions. A number of inflammatory conditions involve antibodies in a pivotal role. It is particularly suprising that the invention is able to suppress inflammatory conditions involving such disease mediating antibodies, even where the disease causing antibodies are still present in the system of the subject and bind to a different epitope to the antibody or fragment with modified glycosylation. Thus, the invention does not require competition between the therapeutic and disease causing antibodies for binding, even though dominant suppression is seen. Thus, the invention provides a way to dominantly suppress the diseases or conditions mentioned herein, particularly those mediated by antibodies.

### Antibodies

Any suitable antibody or fragment may be employed in the present invention. The term “antibody”, as referred to herein, includes whole antibodies and any antigen binding fragment (i.e., “antigen-binding portion”) or single chains thereof. In one preferred instance, the antibody or fragment is monoclonal. In another it is polyclonal.

An antibody refers to a protein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region (abbreviated herein as VL) and a light chain constant region.

The variable regions of the heavy and light chains contain a binding domain that interacts with an antigen. The VH and VL regions can be further subdivided into regions of hypervariability, termed complementarity determining regions (CDR), interspersed with regions that are more conserved, termed framework regions (FR). The constant regions of the antibodies may mediate the binding of the immunoglobulin to host tissues or factors, including various cells of the immune system (e.g., effector cells) and the first component (C1q) of the classical complement system.

The antibody, or fragment, has modified glycosylation. That is the antibody or fragment does not have the normal glycosylation of an antibody of that class. The modification of glycosylation may be the result of:

(i) modification of the natural glycosylation; and/or

(ii) the antibody being synthesised without normal glycosylation.

Reference herein to modified glycosylation encompasses an absence of glycosylation as well as the presence of a different glycosyl group to normal or the presence of an alternative chemical entity. In one preferred instance, the modification is the retention of a partial glycosyl group, particularly a GlcNAc residue with or without a linked fucose group. The presence of fucose linked to the core GlcNAc residue of complex biantennary glysoyl groups is a physiological varation. A substantial fraction (approx 30%) of circulating antibodies lack this fucose.

Thus, in one instance, the antibody or fragment lacks full glycosylation. For instance, whilst it may retain a partial glycosyl group, it does not have the full glycosyl group found on the normal antibody. Such a glycosyl group may be referred to as a truncated glycosyl group. The modification may be enzymatic cleavage, in particular leaving a partial or truncated glycosyl group, particularly a GlcNAc residue with or without linked fucosegroup, which may be referred to as a GlcNac(-Fucose) stump.

Modification of glycosylation may have been achieved by any suitable means, for instance chemically or enzymatically and in particular enzymatically. In a particularly preferred instance, the antibody or fragment will have been treated using the enzyme EndoS, including any of the forms of EndoS described herein. Treatment with Endo S leaves only the first N-acetylglucosamine (GlcNAc) residue with a linked fucose group (if a fucose group was originally present), from the usual glycosyl group, as cleavage has occurred between the two core GlcNAc residues. Reference to modification herein encompasses in particular such a modification and in one particularly preferred instance, the antibody or fragment of the invention comprises such a residual group. Any means of achieving such modifications, or the presence of such a group, may be employed whether chemical or enzymatic. In one instance, the modified group is one obtainable by, preferably obtained by, EndoS cleavage.

In a further instance, the sequence of the antibody or fragment may have been altered so that the natural glycosylation of the antibody does not occur or is modified. Alternatively, the antibody or fragment may have been synthesised in a system that does not result in full glycosylation. Thus, it may be that glycosylation has been prevented, for instance through the use of an inhibitor of the enzyme or enzymes responsible for glycosylation or via synthesis in a system where the enzymes are lacking naturally or have been eliminated using such techniques as knocking out encoding genes. In some systems only partial glycosylation may occur because not all of the enzymes necessary for full glycosylation are present.

In normally glycosylated antibodies, the oligosaccharide is typically of the complex biantennary type comprising two core N-acetylglucosamine (GlcNAc) residues, in which a core fucose may or may not be linked to the innermost N-acetylglucosamine (GlcNAc). These glycans are typically located in the interface between the C_{H}2 domains (second constant domain of the heavy chains). In IgG antibodies glycosylation typically occurs in each heavy chain at Asn-297.

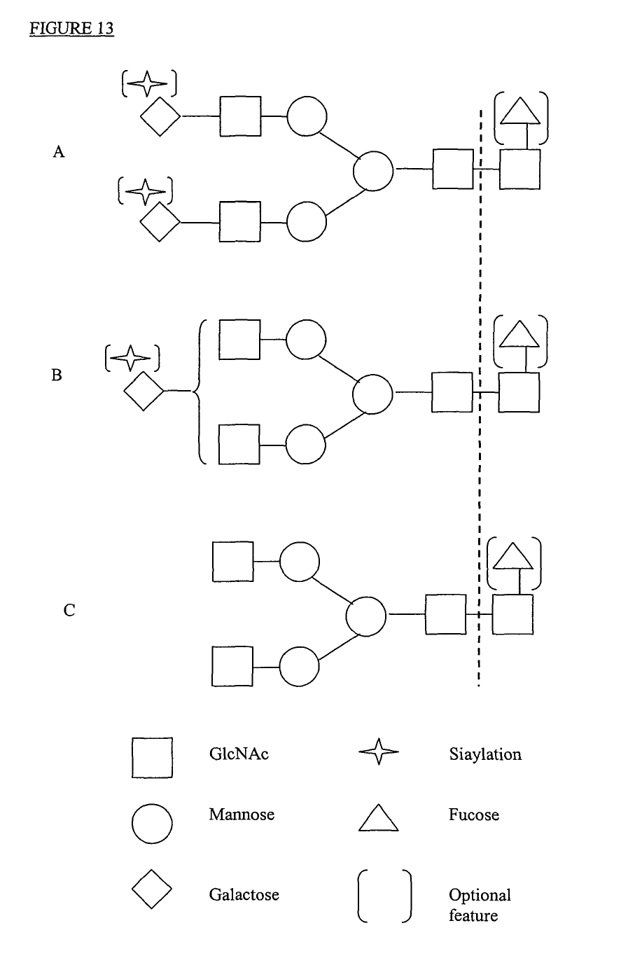

That is, for IgG molecules there is typically a glycosyl group attached to the residue at position 297 of each heavy chain. Examples of the naturally occurring glycosyl groups present at Asn-297 in IgG molecules are shown in FIG. 13. Equivalent or similar glycosylation sites exist in other antibody classes. For example, N-linked glycosylation sites exist in IgM molecules at Asn-171, Asn-332, Asn-395, Asn-402 and Asn-563. All except Asn-402 and Asn-563 are commonly occupied by complex biantennary glycans. N-linked glycosylation sites also exist in IgA molecules at Asn-263 in the CH2 domain, and Asn-459, in IgD molecules at Asn-354, Asn-445 and Asn-496, and in IgE molecules at Asn-140, Asn-168, Asn-218, Asn-365, Asn-371, Asn-383 and Asn-394. The glycosyl groups normally present at these sites are preferably complex biantennary glycans.

The glycosylation in such positions is typically an N-linked glycosyl group, but may be O-linked if a suitable amino acid is present in the relevant position.

In a preferred instance, the antibody or fragment of the invention lacks the normal full glycosylation at a position in one and preferably both of the heavy chains, which position in a normally glycosylated antibody is linked to a complex biantennary glycosyl group comprising two core GlcNAc residues. That is, only a partial or truncated glycosyl chain is linked to said position. In a particularly preferred instance, the antibody is an IgG antibody, and the position is 297 (normally Asn-297) or a corresponding position.

In a particularly preferred instance, the normal glycosyl group is absent and only the first N-acetylglucosamine (GlcNAc) residue remains, with or without the linked fucose group. In other words, the truncated glycosyl group may consist of a single core GlcNAc residue, optionally linked to a fucose residue. Thus, the normal glycosyl chain may have been cleaved by hydrolysing the linkage between the two GlcNAc residues. That represents the cleavage resulting from treatment with EndoS and in a preferred instance the antibody or fragment of the invention has been treated with EndoS to modify the glycosylation of the antibody or fragment. In alternative embodiments, the Asn-297 residue has been substituted or deleted so that glycosylation no longer occurs.

In one instance, the normal glycosyl group is absent and only the first two GlcNAc residues remain, the first GlcNAc residue preferably with its normally linked fucose group (if present). In other words, the truncated glycosyl group may consist of a first core GlcNAc residue linked to a second core GlcNAc residue, wherein the first core GlcNAc residue is optionally linked to a fucose residue. Thus, the normal glycosyl chain may have been cleaved by hydrolysing the linkage between the second GlcNAc residue and mannose.

In one instrance, the normal glycosyl group is absent and only the first two GlcNAc residues and first mannose residue remain. The first GlcNAc residue preferably has its normally linked fucose group (if present). The mannose residue may optionally have be linked to a single further GlcNAc residue, typically by an α1,4 linkage. In other words, the truncated glycosyl group may consist of a first core GlcNAc residue linked to a second core GlcNAc residue linked to a mannose residue, wherein the first core GlcNAc residue is optionally linked to a fucose residue, and/or wherein the mannose residue is optionally linked to a further, single GlcNAc residue. Thus, the normal glycosyl chain may have been cleaved by hydrolysing the linkages between the first mannose residue and the branching mannose residues attached to it. These linkages are typically α1,3 and α1,6 linkages.

In the above instances, one or more GlcNAc residues may be replaced with a N-Acetylgalactosamine (GalNAc) residue.

Alternatively, modification may mean that the glycosyl group is replaced with a different chemical entity. In one instance, the glycosyl group may have been replaced with a monosaccharide or disaccharide, an aliphatic group or compound, an aromatic group or compound, an amino acid or amino acid derivative, a nucleotide or nucleotide derivative, a lipid or lipid derivative, any organic group, or any combination thereof, which results in an antibody or fragment which (a) suppresses an inflammatory condition forming part of the disease or condition to be treated or prevented; and/or (b) displays increased efficacy and/or decreased side effects in comparision to treatment or prevention with the corresponding antibody with unmodified glycosylation. Such an antibody or fragment may typically be an agonist to an antibody or fragment which has modified glycosylation such that the normal glycosylation has been replaced with a truncated glycosyl group consisting of a single core GlcNAc residue, optionally linked to a fucose residue.

In one instance, the modification of glycosylation may mean that the antibody may be unable to activate complement. In a further instance, the antibody or fragment may modify Fc receptor binding. In some embodiments, the antibody or fragment may modify both functions. In some instances, the antibody or fragment may retain the ability to bind to inhibitory FcR but lack binding to activatory FcR. In others, it may not. In other instances the modification of glycosylation may affect the binding for other unknown receptors.

The antibody may be of any antibody class. For instance, it may be IgA, IgD, IgE, IgM or IgG. In an especially preferred embodiment the antibody, or fragment, is IgG or a fragment thereof. The antibody may be any subtype, for instance it may be any of IgG₁, IgG₂, IgG₃ or IgG₄. The antibody, may in some instances be monomeric, dimeric or pentameric, in a particularly preferred instance it may be monomeric.

Preferably, the antibody is one in which an amino acid position in one or both heavy chains is normally linked to a complex biantennary glycosyl group comprising two core GlcNAc residues. Preferably, the amino acid normally occurring at this position is retained in one or both heavy chains, but the glcosyl group attached to one or both is modified. In a preferred instance, the antibody or fragment of the invention lacks the normal full glycosylation at the said position in one and preferably both of the heavy chains. That is, only a partial or truncated glycosyl chain is linked to the position.

The amino acid normally present in the position is typically Asn, but may be any alternative amino acid to which a glycosyl group can be linked, or may be substituted for such an alternative amino acid. Suitable amino acids include Ser, Thr or Asp. Where Ser or Thr are present, the linked glycosyl group may typically be O-linked rather than N-linked. In such instances, one or more GlcNAc residues, typically at least the first core GlcNAc residue, may be replaced with a GalNAc residue.

As explained above, the residue at position 297 (normally Asn-297) in IgG molecules is a preferred example of a position which in a normally glycosylated antibody is linked to a complex biantennary glycosyl group comprising two core GlcNAc residues. Thus, for example, the Asn residue at position 297 in IgG molecules may be replaced with a different amino acid such as Ser, Thr or Asp. Hence the modification in the case of IgG may be N297S or N297T or N297D.

Alternatively, the glycosyl group may be linked to a different position relative to a normally glycosylated antibody, provided that it is possible to link a glycosyl group to the different position. That is, the modified glycosylation of the present invention may be linked to a position corresponding to, but not identical to, a glycosylated position in a normally glycosylated antibody. For example, in IgG molecules, the modified glycosylation may be linked to any one of positions 287 to 296, or 298 to 308, provided that a suitable amino acid is present at said position. Suitable amino acids include in particular Asn, Asp, Ser and Thr. Thus, as a specific example, an Asn residue at position 289 (Asn-289) may be considered to be in a position corresponding to Asn-297.

As used herein, the term “epitope” generally refers to the site on a target antigen that is recognised by an antibody. It may be a short peptide derived from or as part of a protein. However the term is also intended to include peptides with glycopeptides and carbohydrate epitopes. A single antigenic molecule may comprise several different epitopes. Epitopes can be identified from knowledge of the amino acid and corresponding DNA sequences of the peptide, as well as from the nature of particular amino acids (e.g., size, charge, etc.) and the codon dictionary, without undue experimentation. See, e.g., Ivan Roitt, Essential Immunology, 1988; Janis Kuby, Immunology, 1992 e.g., pp. 79-81. In one instance, the binding epitope of a disease causing antibody may be determined in the subject to be treated.

The location of an epitope may be identified by routine methods. For example, the general location of an epitope may be determined by assessing the ability of an antibody to bind to different fragments or variant polypeptides derived from the target. The specific amino acids within the target that make contact with an antibody may also be determined using routine methods. For example, the antibody and target molecule may be combined and the antibody/target complex may be crystallised. The crystal structure of the complex may be determined and used to identify specific sites of interaction between the antibody and its target.

An antibody of the invention is preferably capable of binding to its target with an affinity that is at least two-fold, 10-fold, 50-fold, 100-fold or greater than its affinity for binding to another non-target molecule.

An antibody of the invention may, for instance, be a monoclonal antibody or a polyclonal antibody. In one preferred embodiment, an antibody of the invention is a monoclonal antibody. An antibody of the invention may be a chimeric antibody, a CDR-grafted antibody, a human or humanised antibody or an antigen-binding portion of any thereof. For the production of both monoclonal and polyclonal antibodies, the experimental animal is suitably a mammal such as a goat, rabbit, rat or mouse.

Polyclonal antibodies are antibodies that are derived from different B cell lines. A polyclonal antibody may comprise a mixture of different immunoglobulin molecules that are directed against a specific antigen. The polyclonal antibody may comprise a mixture of different immunoglobulin molecules that bind to one or more different epitopes within an antigen molecule. Polyclonal antibodies may be produced by routine methods such as immunisation of a suitable animal, with the antigen of interest. Blood may be subsequently removed from the animal and the immunoglobulin fraction purified. Modification of glycosylation may then be performed.

Monoclonal antibodies are immunoglobulin molecules that are identical to each other and have a single binding specificity and affinity for a particular epitope. Monoclonal antibodies (mAbs) of the present invention can be produced by a variety of techniques, including conventional monoclonal antibody methodology e.g., the standard somatic cell hybridization technique of Kohler and Milstein (1975) Nature 256: 495, or viral or oncogenic transformation of B lymphocytes. The preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

To generate hybridomas producing monoclonal antibodies of the invention, splenocytes and/or lymph node cells from immunized mice can be isolated and fused to an appropriate immortalized cell line, such as a mouse myeloma cell line. The resulting hybridomas can be screened for the production of antigen-specific antibodies. The antibody secreting hybridomas can be replated, screened again, and if still positive for suitable IgG, the monoclonal antibodies can be subcloned at least twice by limiting dilution. The stable subclones can then be cultured in vitro to generate small amounts of antibody in tissue culture medium for characterization.

Antibody fragments may be employed in the invention. Any suitable antibody fragment may be employed, typically that, when administered, displays reduced side effects, whilst retaining the ability to act as a therapeutic and/or the ability to reduce or ameliorate an immune response in a disease or condition, particularly to suppress an inflammatory condition. Anywhere reference is made herein to the use of an antibody, a functional antibody fragment may also be employed unless the context dictates specifically otherwise.

Preferably, the antibody fragment will retain the residue which is normally linked to a complex biantennary glycosyl group comprising two core GlcNAc residues (for example the Asn-297 residue in IgG), but will have modified glycosylation. Alternatively, it may retain the region comprising said residue, but the specific residue has been substituted or deleted as explained above. The fragment may or may not be capable of binding to antigen. The antibody or fragment may lack an antigen-binding portion. For example, the fragment may be an Fc fragment.

The term “antigen-binding portion” of an antibody refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include a Fab fragment, a F(ab′)₂ fragment, a Fab′ fragment, a Fd fragment, a Fv fragment, a dAb fragment and an isolated complementarity determining region (CDR). Single chain antibodies such as scFv and heavy chain antibodies such as VHH and camel antibodies are also intended to be encompassed within the term “antigen-binding portion” of an antibody. These antibody fragments may be obtained using conventional techniques known to those of skill in the art, and the fragments may be screened for utility in the same manner as intact antibodies.

In one preferred instance, the fragments will lack the ability to bind C1q and/or Fc receptors, but still possess the regions responsible for such binding, preferably the binding of C1q and/or Fc receptors will no longer occur because of the modification of glycosylation, not because of deletion of amino acids responsible for binding.

In one instance, the antibody which is employed is not an antibody fragment.

An antibody of the invention may be prepared, expressed, created or isolated by recombinant means, such as (a) antibodies isolated from an animal (e.g., a mouse) that is transgenic or transchromosomal for the immunoglobulin genes of interest or a hybridoma prepared therefrom, (b) antibodies isolated from a host cell transformed to express the antibody of interest, e.g., from a transfectoma, (c) antibodies isolated from a recombinant, combinatorial antibody library, and (d) antibodies prepared, expressed, created or isolated by any other means that involve splicing of immunoglobulin gene sequences to other DNA sequences.

An antibody of the invention may be a human antibody or a humanised antibody. The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

Such a human antibody may be a human monoclonal antibody. Such a human monoclonal antibody may be produced by a hybridoma which includes a B cell obtained from a transgenic nonhuman animal, e.g., a transgenic mouse, having a genome comprising a human heavy chain transgene and a light chain transgene fused to an immortalized cell.

Human antibodies may be prepared by in vitro immunisation of human lymphocytes followed by transformation of the lymphocytes with Epstein-Barr virus.

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences.

Antibodies of the invention can be tested for binding to the target protein by, for example, standard ELISA or Western blotting. An ELISA assay can also be used to screen for hybridomas that show positive reactivity with the target protein. The binding specificity of an antibody may also be determined by monitoring binding of the antibody to cells expressing the target protein, for example by flow cytometry.

Once a suitable antibody has been identified and selected, the amino acid sequence of the antibody may be identified by methods known in the art. The genes encoding the antibody can be cloned using degenerate primers. The antibody may be recombinantly produced by routine methods.

In one instance, the antibody or fragment employed has two different antibody specificities. For instance, bispecific IgG may be employed.

A “polypeptide” is used herein in its broadest sense to refer to a compound of two or more subunit amino acids, amino acid analogs, or other peptidomimetics. The term “polypeptide” thus includes short peptide sequences and also longer polypeptides and proteins. As used herein, the term “amino acid” refers to either natural and/or unnatural or synthetic amino acids, including glycine and both the D or L optical isomers, and amino acid analogs and peptidomimetics.

In some instances, a mixture of antibodies or antibody fragments may be employed. At least one of the antibodies or fragments in the mixture will have modified glycosylation and in one instance they may all do so. Any embodiment described herein with reference to an antibody or fragment may similarly be performed with a mixture of antibodies or fragments as appropriate. In one instance two, three, four, five or more different antibodies or fragments may be present in the mixture.

### Therapeutic Use of Antibodies of the Invention

An antibody, or antibody fragment, of the invention may be used to treat or prevent any suitable or disease condition. In a particularly preferred instance, the disease or condition comprises, consists essentially of, or consists of, an inflammatory condition, particularly any mentioned herein.

Thus, the invention may be used to suppress an inflammatory condition. Suppression may be eliminating the disease or ameliorating it. One or more symptoms of the condition may be reduced or eliminated. The inflammatory index may be reduced in the subject following application of the invention.

Thus, an antibody or antibody fragment of the invention may be used as an immunosuppressive.

Examples of conditions the invention may be used to treat or prevent include autoimmune disease, post-operative treatment, acquired haemophilia or an alloimmune reaction. Alloimmune conditions include transplant rejection, an immunological reaction upon blood transfusion or an immunological placenta transfer condition. Examples of immunological placenta transfer conditions include haemolytic disease of the fetus and newborn (HDFN) and neonatal alloimmune thrombocytopenia (NAITP).

The inflammatory condition may, for instance, be one mediated, in whole or part, by:
(i) an antibody or antibodies;
(ii) a lymphocyte reaction;
(iii) an innate immune system reaction and/or
(iv) an alloimmune reaction and/or
(v) an autoimmune reaction

In one particularly preferred embodiment of the invention, the invention is employed to treat or prevent a disease characterised by an antibody or antibodies playing a role in the development of the disease or condition. Unexpectedly, in diseases or conditions mediated, in whole or part, by antibodies, administration of an antibody or fragment with modified glycosylation is effective even though the antibodies responsible for the disease or condition are still present in the system of the subject in normally glycosylated form. Thus, not only does the invention suppress the inflammatory condition,it can do so even though the antibodies causing, or contributing, to the condition are still present.

Furthermore, in such embodiments, it has surprisingly demonstrated that the antibodies with modified glycosylation do not have to be against the same epitope as the disease-mediating antibody to suppress an antibody mediated disorder. Thus, the invention may be used to suppress immune responses without having to entail direct competition of the administered antibody for binding with an antibody involved in disease.

In one preferred instance, the antibody or antibody fragment binds a disease related antigen. In other embodiments, it does not. Suprisingly, the antibody or antibody fragment does not have to bind the same epitope as the antibody mediating the disease or condition. Thus, the antibody or fragment employed may, for instance, be:
(i) an antibody or fragment recognising the same epitope as the disease mediating antibody;
(ii) an antibody or fragment recognising a different, but overlapping epiotpe, to the disease mediating antibody;
(iii) an antibody or fragment recognising a different, non-overlapping epitope on the same antigen as the disease mediating antibody; or
(iv) an antibody or fragment recognising a different antigen to the disease mediating antibody;
(v) an antibody or fragment which is not capable of binding antigen.

In one preferred instance, (iv) may be the case. In some instances, the different antigen may be one still assocated with the disease, in another it may be antigen which is not associated with the disease. In some instances, the diferent antigen may be one found in the same tissue or tissues as the disease related antigen, whilst in others it may be found in a different tissue.

In one preferred instance, (v) may be the case, and the fragment may be, for example, an Fc fragment.

The antibody or fragment may display an inability to bind the complement factor C1q.

In one instance, the antigen involved in the disease is one involved in tissue degradation involved in the disease. For instance, the antigen may be a cartilage component whose degradation is seen in arthritis.

In another instance, the antibody or fragment employed may bind to the same epitope as a disease mediating antibody. In one instance, the antibody, or fragment, may correspond to the antibody mediating the disease or condition, with the only difference being the modification of glycosylation.

Thus, whilst binding to the same, or an overlapping epitope, to that recognised by a disease or condition mediating antibody is not obligatory, some embodiments of the invention may employ such antibodies or fragments and it may be that competition for binding with the disease mediating antibody does occur. In some embodiments, though the epitope is not the same, or overlapping in linear terms, the epitope bound by the antibody or fragment of the invention may spatially inhibit binding of the disease or condition mediating antibody. Thus both competition for binding with, and spatial inhibition of binding of, the disease or condition mediating antibody may contribute to the invention in some embodiments. In some instances, a mixture of antibodies may be employed, for instance a mixture of at least two of antibody types (i) to (iv) mentioned above, in one case a mixture of such monoclonals.

In other embodiments, the disease or condition is not one mediated by an antibody and the invention applies a general immunosuppressive effect to eliminate or reduce an unwanted immune response.

In one preferred instance, the disease or condition the antibody, or fragment, is administered to treat is one referred to in Table 1. The antigen targeted may, in a further preferred instance, be one referred to in Table 1 below.

In a further instance, the antibody or fragment is a therapeutic antibody which has been used in normally glycosylated form previously, but not in a form with modified glycosylaion. The fact that the antibody has modified glycosylation may increase the potency of the antibody in comparison to the normally glycosylated form. Alternatively, or additionally, it may reduce a side-effect seen with the glycosylated form. These effects may also be mediated by the effect of the antibody on the immune system, thus the various aspects of the invention are linked by the modification of glycosylation altering the effect of the antibody on the immune system, either in terms of suppressing a pre-existing disease or condition, in terms of increasing potency or in terms of reducing or eliminating a side effect.

The therapeutic antibodies may be against any suitable therapeutic target. For instance, the antibody may be one against a cancer antigen, an autoantigen, an antigen associated with allergy, an antigen from a pathogen (for instance from a pathogenic bacteria, virus or fungus) or from a parasite. The antibody may be one against a mediator of such conditions, for instance against a molecule important in the development of autoimmune conditions or allergy. For instance, the antibody may be one against a cytokine or an interleukin. The antibody or fragment may be one against a TNF, such as TNF-alpha.

Examples of possible targets include, but are not limited to, C5, CBL, CD147, IL-8, VLA-4, CD11a, CD18, VEGF, anti-Id, ICAM-1, CD2, EGFR, TGF-beta 2, TNF-alpha, Her2/neu, F gp, CD11/18, CD14, ICAM-3, CD80, CD4, CD23, beta2-integrin, alpha4beta7, CD52, HLA DR, CD22, CD64 (FcR), TCR alpha beta, Hep B, CA 125, EpCAM, gp120, gpIIbIIa, CD20, IL-4, IL-5, IL-6, CD33, cadherin 11 and VNRintegrin. In one instance, the antibody or antibody fragment may be one against at least one of CD3, CD20, GPIIb/IIIa, TNF, CD25, EGF-R, CD25, Her2/neu, RSV, CD33, CD52, CD11a, IL-6R and VLA4. The antibody may be one against GM-CSF.

A number of antibodies available in glycosylated form, may, in light of the invention now be provided with modified glycosylation in order to reduce or eliminated unwanted effects of the antibodies. Possible antibodies that the invention may be applied to include, but is not limited to, 5G1.1, 5G1.1-SC, ABX-CBL, ABX-IL8, AD-159, AD-439, Antegren, Anti-CD11a, Anti-CD18, Anti-LFA1, Anti-VEGF, Antova, BEC2, BIRR-1, BTI-322, C225, CAT-152, CDP571, CDP850, Corsevin M, D2E7, Herceptin, HNK20, Hu23F2G, IC14, ICM3, IDEC-114 and IDEC-131. A further list of possible antibodies which may be provided in a form with modified glycosylation include Rituxan/MabThera (rituximab), Remicade (infliximab), Avastin (bevacizumab), Herceptin (trastuzumab), Synagis (palivizumab), Erbitux (cetuximab), Humira (adalimumab), Simponi (golimumab) and Actemra (tocilizumab). Additional antibodies that the invention may be applied to include Orthoclone, Zevalin, Bexxar, ReoPro, Simulect, zenapax, Mylotarg, Campath, Xolair, Raptiva, Avastin, Actemra and Tysabri.

TABLE 1

| DISEASE | AUTOANTIGENS |
|---|---|
| Addison's disease | Steroid 21-hydroxylase, 17 alpha-Hydroxylase |
| | (17OH) and side-chain-cleavage enzyme |
| | (P450scc), Thyroperoxidase, thyroglobulin and |
| | H+/K(+)− |
| Anti-GBM glomerulonephritis (related to | Anti-glomerular basement membrane (anti-GBM): |
| Goodpasteur) | noncollagenous (NC1) domains of the |
| | alpha3alpha4alpha5(IV) collagen |
| Anti-neutrophil cytoplasmic antibody-associated | Myeloperoxidase, proteinase 3 |
| vasculitides (ANCA associated | |
| vasculitis)(Wegener granulomatosis, Churg- | |
| Strauss syndrome, microscopic polyangiitis) | |
| Anti-phospholipid antibody syndrome (APS) | Negatively-charged phospholipids complexed with |
| | phospholipid binding plasma proteins (e.g. |
| | beta2GPI), cardiolipin, beta2-glycoprotein I, and |
| | (beta2GPI) |
| Autoimmune bullous skin diseases (Pemphigus). | IgG against keratinocytes. Specific target is |
| Pemphigus foliaceus (PF), fogo selvagem | desmoglein (Dsg) 1 (desmosomal |
| (FS)(endemic form), pemphigus vulgaris (PV) | Cadherins) |
| Autoimmune hemolytic anemia (AIHA) | Self-antigens on red-blood-cells |
| Autoimmune hepatitis (AIH) | Actin, antinuclear antibody (ANA), smooth muscle |
| | antibody (SMA), liver/kidney microsomal antibody |
| | (LKM-1), anti soluble liver antigen (SLA/LP) and |
| | anti-mitochondrial antibody (AMA), CYP2D6, |
| | CYP2C9-tienilic acid, UGT1A, CYP1A2, |
| | CYP2A6, CYP3A, CYP2E1, CYP11A1, CYP17 |
| | and CYP21 |
| Autoimmune neutropenia (AIN) | FcgRIIIb |
| Bullous pemphigoid (BP) | Hemidesmosomal proteins BP230 and BP180 (type |
| | XVII collagen), laminin 5, the alpha6 subunit of the |
| | integrin alpha6beta4 and p200 |
| Celiac disease | transglutaminase 2 (TG2), transglutaminase 3, |
| | actin, ganglioside, collagen, calreticulin and |
| | zonulin, thyroid, endocrine pancreas, anti-gastric |
| | and liver, anti-nuclear constituents, anti-reticulin, |
| | actin, smooth muscle, calreticulin, desmin, |
| | collagens, bone, anti-brain, ganglioside, neuronal, |
| | blood vessel |
| Chronic utricaria | Alpha-subunit of the high-affinity IgE receptor, IgE |
| Complete congenital heart block (CCHB) | Ro (Sjögens syndrome antigen A (SSA)), La |
| | (Sjögens syndrome antigen B(SSB)) |
| Diabetes type 1A (T1DM) | Islet cell autoantibodies (ICA), antibodies to insulin |
| | (IAA), glutamic acid decarboxylase (GAA or |
| | GAD), protein tyrosine phosphatase (IA2 or |
| | ICA512), Insulinoma Associated Peptide-2. The |
| | number of antibodies, rather than the individual |
| | antibody, is thought to be most predictive of |
| | progression to overt diabetes. |
| Essential mixed cryoglobulinemia | Essential mixed cryoglobulinemia antigens |
| Goodpasture's syndrome (also known as | alpha3(IV) collagen (=Goodpasture antigen) |
| Goodpasture's disease and | |
| anti-glomerular basement membrane disease | |
| Graves'disease (Basedow's disease), includes | Thyrotropin receptor (TSHR) Thyroid peroxidase |
| Goitre and hyperthyroidism, infiltrative | (TPO) |
| exopthalmos and infiltarative dermopathy. | |
| Guillain-Barré syndrome (GBS). Acute | Gangliosides GM1, GM1b, GD1a, and GalNAc- |
| inflammatory demyelinating polyneuropathy | GD1a, glycosphingolipid, myelin proteins PMP22 |
| (AIDP), acute motor axonal neuropathy (AMAN) | and P0 |
| Hemophilia - Acquired FVIII deficiency | Factor VIII |
| Idiopathic thrombocytopenic purpura (ITP) | Platelet glycoprotein (GP) IIb-IIIa and/or GPIb-IX |
| Lambert-Eaton myasthenic syndrome (LEMS) | voltage gated calcium channels |
| Mixed Connective Tissue Disease (MCTD) | IgG directed against the spliceosome, U1-snRNP |
| Multiple Myeloma | Multiple Myeloma antigens |
| Myasthenia gravis | Acetylcholine receptors (AchR), muscle-specific |
| | kinase (MuSK) |
| Myocarditis, dilated cardiomyopathy | heart-reactive autoantibodies against multiple |
| (DCM)(congestive cardiomyopathy) | antigens e.g. cardiac myosin |
| Primary biliary cirrhosis (PBC) | pyruvate dehydrogenase complex (PDC)-E2 and |
| | other members of the oxaloacid dehydrogenase |
| | family, Glycoprotein-210, p62, sp100 |
| Primary Progressive Multiple Sclerosis (PPMS) | Myelin oligodendrocyte glycoprotein |
| | (MOG), Myelin proteolipid protein (PLP), |
| | transketolase (TK), cyclic nucleotide |
| | phosphodiesterase type I (CNPase I), |
| | collapsin response mediator protein 2, tubulin |
| | beta4, neurofascin |
| Rheumatic heart disease (RHD), (Rheumatic | Cardiac myosin |
| fever) | |
| Rheumatoid Arthritis (RA) | Type II collagen, citrullin (-ated proteins (e.g. (fibrinogen, |
| | vimentin, filaggrin, type II collagen, enolase)), G6PI, RFs (anti- |
| | Fc/IgG), Vimentin, and cytokeratin |
| Sjögren Syndrome (SS) | Ro (Sjögens syndrome antigen A (SS-A)), La (Sjögens |
| | syndrome antigen B(SS-B)), p80 coilin, antinuclear antibodies, |
| | anti-thyroid, anti-centromere antibodies (Raynaud's |
| | phenomenon), anti-carbonic anhydrase II (distal renal |
| | tubular acidosis), anti-mitochondrial antibodies (liver |
| | pathology), cryoglobulins (evolution to non-Hodgkin's |
| | lymphoma). alpha- and beta-fodrin, islet cell autoantigen, |
| | poly(ADP)ribose polymerase (PARP), NuMA, Golgins, NOR- |
| | 90, M3-muscarinic receptor |
| SLE including Lupus nephritis | Autoantibodies to nuclear constituents (e.g. dsDNA and |
| | nucleosomes), dsDNA, PARP, Sm, PCDA, rRNA Ribosome P |
| | proteins, C1q |
| Stiff-person syndrome (SPS) | glutamic acid decarboxylase (GAD), amphiphysin. |
| Systemic sclerosis (scleroderma) | DNA-topoisomerase I (Scl-70), U3 snRNP, U2 snRNP, 7-2 |
| | RNP, NOR-90, centromere-associated proteins, and nucleolar |
| | antigens, Anti-Th/To, Anti-RNA |
| | polymerase I/III, Anti-PDGF receptor, Anti-fibrillin-1, M3- |
| | muscarinic receptor, |
| Transplant rejection | Transplant rejection antigens |

The therapeutic antibody may be to treat any disease or condition where antibody treatment is appropriate. The disease or condition may be any of those mentioned herein. The modification of glycosylation may result in prolonging the effect of the therapeutic antibody. It may redirect the therapeutic effect of the antibody.

In one particularly preferred embodiment, the antibody or fragment is one that binds a red blood cell antigen. For instance, an antigen found on the surface of a red blood cell, such as a red blood cell structural protein, a blood group protein or a red blood cell carbohydrate. The antigen may be one involved in AIHA, for instance, one of the four membrane proteins that acts as an autoantigen in AIHA. The therapeutic antibody or fragment may be against an antigen involved in a haemolytic disease of the newborn or one involved in SLE

In one particularly preferred embodiment, the antibody or fragment is one against a Rhesus antigen, such as the RhD, Cc and Ee antigen. Thus, preferably, the antibody is against Rhesus D antigen, hence in a particularly preferred embodiment, the antibody is anti-D. The antibody may be one administered to treat an immune reaction by a mother against a foetus. In one particularly preferred instance, the antibody may be one of Rhesonativ, Octapharma, Lachen, Schweiz or Diamed, but where the antibody has modified glycosylation.

Intravenous preparations of immune globulin (IVIG) are used to treat a variety of conditions. The invention may be applied to IVIG and to any disease or condition mentioned herein. Hence the invention provides IVIG where the antibodies present have modified glycosylation. Typically, IVIG is prepared using blood from a plurality of people and in one embodiment, the IVIG provided by the invention has been so provided. Conditions which may be treated using the IVIG preparations of the invention include idiopathic thrombocytopenic purpura (ITP), primary immunodeficiency, autoimmune thrombocytopenia, the vascular disorder Kawaskai disease, hematopoietic stem cell or bone marrow transplantation, particularly in patients older than 20 years, chronic B-cell lymphocytic leukemia, prevention of graft versus host disease in transplant patients, and paediatric HIV-1 infection.

IVIG preparations of the invention may also be employed to treat aplastic anemia, red blood cell aplasia, autoimmune hemolytic anemia, hemolytic disease of the newborn, patients with acquired clotting factor inhibitors, acquired von Willebrand disease, immune-mediated neutropenia (deficiency of polysegmented white blood cells), pemphigoid disorders, refractoriness to platelet transfusions, blood transfusion reactions or consequences, Graves' ophthalmopathy, pretibial myxedema, multiple sclerosis, CIDP, and various systemic autoimmune rheumatological conditions including rheumatoid arthritis, dermatomyositis and systemic lupus erythematosus (SLE). In patients at risk for infectious diseases because of compromised immune systems such as patients with burns, trauma, low birth weight or HIV infection. IVIG preparations of the invention may be used to treat any severe autoimmune disease. IVIG preparations of the invention may be used to treat carbon monoxide poisoning. IVIG may also be employed to treat Guillain-Barre syndrome, polyneuropathy, vasculitis, systemic lupus erythematosus, antiphospholipid syndrome and myaesthenia gravis. IVIG may be used to help prevent, or reduce the risk of, miscarriage. It may be used to treat hypogammaglobulinemia, pemphigus, polymyositis (PM), dermatomyositis (DM), Wegener's granulomatosis (WG), Churg-Strauss syndrome and chronic inflammatory demyelinating polyneuropathy (CIDP).

Any of the conditions mentioned herein in relation to IVIG, may also be the subject of treatment using any of the antibodies or fragments of the invention.

The invention may result in reduction of one or more parameters forming part of an inflammatory condition. For instance, reduced Fc receptor binding may be seen. A reduction of opsonisation may occur. Reduced Fc receptor mediated phagocytosis may be seen, such as reduced Fc receptor mediated erythrophagocytosis. In some instances, reduced complement activation may be seen and a drop in activation of the oxidative burst triggered by Fc receptors may also occur. Cytokine released trigged by Fc receptor binding may also be altered or changed, for instance, IL-8 release may be reduced, particularly where the antibody is one against RBCs or IVIG is administered.

In order to assess the reduction or elimination of a particular parameter any suitable technique may be employed, including via employing the assays described in the Examples of the present application. For instance, those for assessing phagocytosis, quantification of cell bound antibody or fragment, monocyte monolayer assays (MMAs), quantification of phagocytosis of RBC via measuring RBC. ELISA, ELISPOT, chemiluminescence and Western blot based assays may be employed. Cytokine release may be measured. Complement related parameters measured, such as complement uptake, in particular C1q. In one particularly preferred embodiment, Fc receptor mediated phagocytosis of RBC will be measured, particularly where the antibody or fragment is anti-D. Any of the assays mentioned herein may be peformed in vitro. Assessment may be made during treatment, for instance by obtaining samples during treatment at particular timepoints and performing such assays on the samples obtained. Appropriate controls may also be used, such as a comparison with a fully glycosylated antibody. Such assessment may be performed for both where the aim is to treat or prevent a disease or condition or alternatively to reduce a side effect or increase potency.

In the case of an antibody against an RBC antigen, reduced phagocytosis and removal of RBCs by cells such as monocytes or macrophages may be seen. This may, in some instances, be evidenced by reduced hemoglobin accumulation in phagocytic cells. In one preferred instance, RBC lysis may be reduced. Such lysis may be reduced extravascularly or intravascularly or both.

Undesirable hemolysis is found as both an undesirable side effect in both administration of anti-D and also in administration IVIG. Thus, in both situations the undesirable haemolysis may be reduced and treatment improved. In one particularly preferred embodiment, therefore the invention is applied to reduce hemolysis seen in antibody based treatments, particularly for both anti-D and also IVIG. Reduced hemolysis may result in reduced intravascular coagulation.

In one preferred instance, the disease or condition the antibody, or fragment, is administered to treat is one referred to in Table 1. The antigen targeted may, in a further preferred instance, be one referred to in Table 1.

### Modification of Glycosylation

### Enzymatic Modification

In one instance, the glycosylation of an antibody may be enzymatically modified. Any suitable enyzme may be employed to modify glycosylation of an antibody or fragment. In one particularly preferred instance, the enzyme used for the modification is an EndoS. Thus, for any of the embodiments mentioned herein, preferably the modification to the glycosylation is that made by EndoS, or that obtainable by EndoS treatment. However, any suitable enzyme may be employed.

The EndoS polypeptide is preferably S. pyogenes EndoS, or a variant or fragment of S. pyogenes EndoS which retains immunoglobulin endoglycosidase activity. The variant may be an EndoS polypeptide from another organism, such as another bacterium. The bacterium is preferably a Streptococcus, such as Streptococcus equi, Streptococcus zooepidemicus or, preferably, Streptococcus pyogenes. Alternatively, the variant may be from Corynebacterium pseudotuberculosis, for example the CP40 protein; Enterococcus faecalis, for example the EndoE protein; or Elizabethkingia meningoseptica (formerly Flavobacterium meningosepticum), for example the EndoF₂ protein.

The EndoS polypeptide may, for instance, comprise:

(a) the amino acid sequence of SEQ ID NO: 1;

(b) a variant thereof having at least 50% identity to the amino acid sequence of SEQ ID NO: 1 and having immunoglobulin endoglycosidase activity; or

(c) a fragment of either thereof having immunoglobulin endoglycosidase activity.

In one preferred instance, the EndoS polypeptide comprises, or consists of, the sequence of SEQ ID NO: 1. SEQ ID NO: 1 is the sequence of the mature form of EndoS, without the signal sequence, and corresponds to amino acids 37 to 995 of SEQ ID NO: 2.

The EndoS polypeptide employed may additionally include a signal sequence. Accordingly, the EndoS polypeptide employed may comprise:

(a) the amino acid sequence of SEQ ID NO: 2;

(b) a variant thereof having at least 50% identity to the amino acid sequence of SEQ ID NO: 2 and having immunoglobulin endoglycosidase activity; or

(c) a fragment of either thereof having immunoglobulin endoglycosidase activity.

The EndoS polypeptide may consist of the sequence shown in SEQ ID NO: 2.

Variant polypeptides are those for which the amino acid sequence varies from that in SEQ ID NO: 1 or SEQ ID NO: 2, but which retain the same essential character or basic functionality as EndoS. The variant polypeptides may therefore display antibody endoglycosidase activity, particularly IgG endoglycosidase activity. Typically, polypeptides with more than about 50%, 55% or 65% identity, preferably at least 70%, at least 80%, at least 90% and particularly preferably at least 95%, at least 97% or at least 99% identity, with the amino acid sequence of SEQ ID NO: 1 or SEQ ID NO: 2 are considered variants of the protein. Such variants may include allelic variants and the deletion, modification or addition of single amino acids or groups of amino acids within the protein sequence, as long as the peptide maintains the basic functionality of EndoS. The identity of variants of SEQ ID NO: 1 or SEQ ID NO: 2 may be measured over a region of at least 100, at least 250, at least 500, at least 750, at least 800, at least 850, at least 900, at least 950, at least 995 or more contiguous amino acids of the sequence shown in SEQ ID NO: 1 or SEQ ID NO: 2, or more preferably over the full length of SEQ ID NO: 1 or SEQ ID NO: 2.

Amino acid identity may be calculated using any suitable algorithm. For example the UWGCG Package provides the BESTFIT program which can be used to calculate homology (for example used on its default settings) (Devereux et al (1984) Nucleic Acids Research 12, 387-395). The PILEUP and BLAST algorithms can be used to calculate homology or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The variant sequences typically differ by at least 1, 2, 3, 5, 10, 20, 30, 50, 100 or more mutations (which may be substitutions, deletions or insertions of amino acids). For example, from 1 to 100, 2 to 50, 3 to 30 or 5 to 20 amino acid substitutions, deletions or insertions may be made. The modified polypeptide generally retains activity as an immunoglobulin-specific endoglycosidase. The substitutions are preferably conservative substitutions, for example according to the following Table. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Variants of the amino acid sequence of SEQ ID NO: 1 preferably contain residues 191 to 199 of SEQ ID NO: 1, i.e. Leu-191, Asp-192, Gly-193, Leu-194, Asp-195, Val-196, Asp-197, Val-198 and Glu-199 of SEQ ID NO: 1 (which correspond to residues 227 to 235 of SEQ ID NO: 2, i.e. Leu-227, Asp-228, Gly-229, Leu-230, Asp-231, Val-232, Asp-233, Val-234 and Glu-235 of SEQ ID NO: 2). These amino acids constitute a perfect chitinase family 18 active site, ending with glutamic acid. The glutamic acid in the active site of chitinases is essential for enzymatic activity. Most preferably, therefore, the variant of SEQ ID NO: 1 contains Glu-199 of SEQ ID NO: 1 and the variant of SEQ ID NO: 2 contains Glu-235 of SEQ ID NO: 2. The variant of SEQ ID NO: 1 may contain residues 191 to 199 of SEQ ED NO: 1 having one or more conservative substitutions, provided that the variant contains Glu-199 of SEQ ID NO: 1. Alternatively, the variant of SEQ ID NO: 2 may contain residues 227 to 235 of SEQ ID NO: 2 having one or more conservative substitutions, provided that the variant contains Glu-235 of SEQ ID NO: 2.

The fragment of the EndoS polypeptide used in the invention is typically at least 10, for example at least 20, 30, 40, 50 or more amino acids in length, up to 100, 200, 250, 300, 500, 750, 800, 850, 900, 950, 995 amino acids in length, as long as it retains the IgG endoglycosidase activity of EndoS. Those fragment lengths in one preferred instance are in relation to fragments from the sequence of SEQ ID No:1. In some instances, longer EndoS enzymes exist and hence longer fragments from them may be employed. For instance, a fragment may be at 1000, 1250, 1500 or 1750 amino acids in length.

Preferably, the fragment of the EndoS polypeptide used in the invention encompasses residues 191 to 199 of SEQ ID NO: 1, i.e. Leu-191, Asp-192, Gly-193, Leu-194, Asp-195, Val-196, Asp-197, Val-198 and Glu-199 of SEQ ID NO: 1 (residues 227 to 235 of SEQ ID NO: 2, i.e. Leu-227, Asp-228, Gly-229, Leu-230, Asp-231, Val-232, Asp-233, Val-234 and Glu-235 of SEQ ID NO: 2). A preferred fragment of SEQ ID NO: 2 consists of amino acids 37 to 995 of SEQ ID NO: 2, i.e. SEQ ID NO: 1, which corresponds to the form of EndoS secreted from S. pyogenes after removal of the signal peptide. Another preferred fragment of the invention consists of amino acids 1 to 409 of SEQ ID NO: 1 (amino acids 37 to 445 of SEQ ID NO: 2), which corresponds to the enzymatically active a-domain of EndoS generated by cleavage by the streptococcal cysteine proteinase SpeB.

The EndoS used in the invention may be chemically modified, e.g. post-translationally modified. For example, they may be glycosylated, phosphorylated or comprise modified amino acid residues. They may be modified by the addition of histidine residues to assist their purification or by the addition of a signal sequence to promote insertion into the cell membrane. Such modified polypeptides fall within the scope of the term “polypeptide” used herein.

The EndoS for use in accordance with the invention display immunoglobulin endoglycosidase activity, and in particular IgG endoglycosidase activity. Preferably, the polypeptide hydrolyzes the β-1,4-di-N-acetylchitobiose core of the asparagine-linked glycan of IgG. Preferably the activity may be specific for IgG. The endoglycosidase activity may be determined by means of a suitable assay. For example, a test polypeptide may be incubated with an antibody, typically IgG, at a suitable temperature, such as 37° C. The starting materials and the reaction products may then be analysed by SDS PAGE. Typically, the molecular mass of the IgG heavy chain is reduced by approximately 3 kDa if the test polypeptide has endoglycosidase activity. Another assay for determining whether a test polypeptide has antibody endoglycosidase activity is by detection of glycosylated antibody using Lens culinaris agglutinin lectin (LCA), optionally using horseradish peroxidase and peroxidase substrate. Typically, the carbohydrate signal is reduced if the test polypeptide has antibody endoglycosidase activity. Another assay for determining whether a test polypeptide has antibody endoglycosidase activity is by incubation of a test polypeptide with purified Fc fragments followed by reduction of the sample with 10 mM dithiotreitol and mass spectroscopy (MALDI-TOF) analysis. Typically, the mass of monomeric IgG Fc is reduced by 1417±14 Da if the test polypeptide has antibody endoglycosidase activity.

The endoglycosidase activity of the polypeptides can be further characterised by inhibition studies.

The endoglycosidase activity of the polypeptide may be, in a preferred instance, be IgG-specific in that the polypeptide may not degrade the other classes of Ig, namely IgM, IgA, IgD and IgE, when incubated with these immunoglobulins under conditions that permit cleavage of IgG. Where the subject is a human, the EndoS polypeptide may be capable of hydrolyzing human IgG. EndoS may be used to hydrolyze human IgG of all four subclasses (IgG₁₋₄). In preferred embodiments, the EndoS polypeptide has the ability to hydrolyze human, Rhesus monkey, mouse, rat, rabbit, horse, goat, dog and swine antibodies, particular such IgG.

Glycosyl modifying enzymes for use in the present invention may be isolated from any suitable organism that expresses an EndoS polypeptide or a variant of an EndoS polypeptide. Typically, the EndoS polypeptide is isolated from suitable EndoS expressing strains of Streptococcus, preferably strains of S. pyogenes. Suitable organisms and strains may be identified by a number of techniques. For example, S. pyogenes strains may initially be tested for the presence an ndoS gene. Polynucleotide primers or probes may be designed based on, for example, SEQ ID NOs: 1, 2 or 3. The presence of the ndoS gene can then be verified by PCR using such primers or by hybridisation of probes to genomic DNA of the S. pyogenes strain.

Streptococcal strains expressing active EndoS or a variant thereof can be identified by assaying for IgG endoglycosidase activity in the culture supernatant or by immunodetection using antibodies directed towards EndoS. The Streptococcal strains that have been verified as expressing active EndoS are the S. pyogenes M1 serotype strains AP1 and SF370, the S. equi strain 4047 and the S. zooepidermicus strain H70. In addition, the ndoS gene is found in the following S. pyogenes strains: M1 serotype strains SSI-1 and MGAS5005, M2 serotype strain MGAS10270, M3 serotype strain MGAS315, M4 serotype strain MGAS10750, M5 serotype strain Manfredo, M6 serotype strain MGAS10394, M12 serotype strain MGAS9429, M18 serotype strain MGAS8232, M28 serotype strain MGAS6180 and M49 serotype strain 591.

Isolation and purification of EndoS from an expressing S. pyogenes culture, or from cultures of other cells expressing EndoS is typically on the basis of antibody endoglycosidase activity, particularly IgG activity. Preferably the purification method involves an ammonium sulphate precipitation step and an ion exchange chromatography step. According to one method, the culture medium is fractionated by adding increasing amounts of ammonium sulphate. The amounts of ammonium sulphate may be 10 to 80%. Preferably the culture medium is fractionated with 50% ammonium sulphate, and the resulting supernatant is further precipitated with 70% ammonium sulphate. Pelleted polypeptides may then be subjected to ion exchange chromatography, for example by FPLC on a Mono Q column. Eluted fractions may be assayed for IgG endoglycosidase activity and peak activity fractions may be pooled. Fractions may be analysed by SDS PAGE. Fractions may be stored at −80° C.

In an alternative method to purify EndoS, EndoS without the signal sequence (i.e. having the sequence of SEQ ID NO: 1) is expressed in Escherichia coli using GST Gene Fusion System (Amersham-Pharmacia Biotech, Uppsala, Sweden). A 2929 base pair PCR product covering bases 304 to 3232 of the ndoS sequence is amplified from S. pyogenes genomic DNA using primers 5′-ACT-GGG-ATC-CCG-GAG-GAG-AAG-ACT-3′ with a BamHI site (underlined) and 5′-TTA-ATC-TCG-AGG-TTG-CTA-TCT-AAG-3′ with an XhoI site (underlined). This fragment is digested with BamHI and XhoI and ligated into the pGEX-5X-3 generating plasmid pGEXndoS that is used to transform E. coli BL21(DE3)pLys. pGEXndoS/BL21(DE3)pLys is induced with 0.1 mM isopropyl β-D-thiogalactopyranoside. After induction, bacteria are lysed using BugBuster™ (Novagen) and the GST-EndoS fusion protein is purified on Glutathione-Sepharose®. The GST tag is removed using factor Xa according to protocols (Amersham-Pharmacia Biotech), and residual factor Xa is removed using Xarrest™-agarose (Novagen). This results in a preparation of recombinant EndoS (rEndoS) that is homogenous as assessed by SDS-PAGE and Western blot using EndoS-specific antibodies. Prior to in vivo experiments protein samples are sterile-filtered through a 0.2 μm filter (Millipore). Purified EndoS protein is stored at −80° C. in phosphate buffered saline.

EndoS enzymes for use in the invention may also be prepared as fragments of such isolated polypeptides. Further, the EndoS polypeptides may also be made synthetically or by recombinant means. For example, a recombinant EndoS polypeptide may be produced by transfecting mammalian cells in culture with an expression vector comprising a nucleotide sequence encoding the polypeptide operably linked to suitable control sequences, culturing the cells, extracting and purifying the EndoS polypeptide produced by the cells.

A number of side chain modifications are known in the art and may be made to the side chains of the EndoS polypeptides, provided that the polypeptides retain antibody endoglycosidase activity and in particular in relation to IgG.

In one instance, the modification may be performed using an enzyme in isolated form, for instance EndoS. In others the system that the antibody or fragment is being synthesised may also express a glycosyl modifying enzyme to bring about removal or modification of the antibody glycosylation. In particular, a system may be employed which expresses both the antibody or antibody fragment and Endo S. In one instance, the host cell employed may comprise nucleotide sequences that encode and can express both the antibody, or fragment, and the enzyme, in particular Endo S.

In further instances, the enzyme employed may be EndoE. In one instance, any suitable enzyme producing the same, or equivalent, modification to EndoS or EndoE may be employed, in a further instance EndoS or Endo E themselves are employed and in a preferred instance EndoS may be employed. In one instance, Endo F₂ is employed, for instance EndoF₂ from Elizabethkingia meningoseptica. EndoE from Enterococcus faecalis may be employed in one instance. A further enzyme that may be employed in the invention is N-glycosidase F (PNGase F), StrH from Streptococcus pneumoniae or EndoC from group C Streptococci.

In one preferred instance, purified antibodies are incubated with EndoS fused to Glutathione S-transferase (GST) in phosphate buffered saline for one hour at 37° C. The amount of GST-EndoS may be in the range 1-20 μg per mg of antibodies and the final antibody concentration is typically between 0.5 and 50 mg/ml. Subsequently, GST-EndoS can if preferred be removed from the reaction mixture by passing three times through a 0.5-5 ml Glutathione-Sepharose column. Alternatively, antibodies can be purified from the reaction mixture by standard affinity chromatography methods.

In another instance, purified antibodies are incubated with the enzymes EndoS, EndoE, EndoC, EndoF2, or StrH in phosphate buffered saline for 1-20 h at 37° C. The amount of enzyme may typically be in the range 1-100 μg per mg of antibodies and the final antibody concentration is typically between 0.5 and 50 mg/ml. Subsequently, antibodies can be purified from the reaction mixture by standard affinity chromatography methods.

In one instance, the enzyme used has been processed by the cysteine proteinase SpeB, particularly where the enzyme is EndoS.

### Other Modification Methods

In one instance, the glycosylation of an antibody may modified relative to normal glycosylation by starting with an aglycosylated antibody and treating it with appropriate transferase enzymes, for example a GlcNAc-transferase enzyme, in order to produce the desired modified glycosyl group. In a preferred embodiment, only a single GlcNAc group is added. In one embodiment, a fucose may be added.

In one instance, the glycosylation of an antibody may be modified de novo by modification of an antibody producing cell-line (e.g. CHO, NSO, Sp2/0, Pichia pastoris, Lemna) such that it expresses the enzymes needed to produce an antibody with the desired modified glycosyl group. For example, the cell line may be modified to knock out mannose transferase enzymes such that only GlcNAc units may be added, alternatively the cell line may be transfected with only an appropriate GlcNAc-transferase.

### Polynucleotides

An antibody or fragment of the invention may be expressed from a polynucleotide. In one instance, such polynucleotides may be employed to recombinantly express an antibody or fragment of the invention. For instance, an antibody or fragment may be expressed and then the glycosylation modified to produce the antibody or fragment for use in the invention.

Alternatively, the polynucleotide may encode an antibody or fragment altered to lack a glycosylation site so that when expressed the antibody lacks glycosylation. In a further embodiment, the system may be such that the antibody or fragment is produced, but the system lacks the necessary enzymes for glycosylation.

In a further instance, a polynucleotide encoding an antibody lacking the site for glycosylation may be administered therapeutically to a subject to be treated.

In one instance, a single polynucleotide may encode all of the necessary antibody chains. In another instance, the different antibody chains may be encoded by separate polynucleotides.

Typically the polynucleotide employed is DNA. However, the polynucleotide may be an RNA polynucleotide. The polynucleotide may be single or double stranded, and may include within it synthetic or modified nucleotides.

Polynucleotides for use in the invention may be produced recombinantly, synthetically, or by any means available to those of skill in the art. They may also be cloned by standard techniques. The polynucleotides are typically provided in isolated and/or purified form.

In general, short polynucleotides will be produced by synthetic means, involving a stepwise manufacture of the desired nucleic acid sequence one nucleotide at a time. Techniques for accomplishing this using automated techniques are readily available in the art. Reference is made to Sambrook et al. (1989).

The polynucleotides for use in the invention are typically incorporated into a recombinant replicable vector. The vector may be used to replicate the nucleic acid in a compatible host cell. Therefore, polynucleotides for use in the invention may be made by introducing an antibody or antibody fragment encoding polynucleotide into a replicable vector, introducing the vector into a compatible host cell and growing the host cell under conditions which bring about replication of the vector. The host cell may, for example, be an E. coli cell.

Preferably the vector is an expression vector comprising a nucleic acid sequence that encodes an antibody or antibody fragment. Such expression vectors are routinely constructed in the art of molecular biology and may for example involve the use of plasmid DNA and appropriate initiators, promoters, enhancers and other elements, such as for example polyadenylation signals, which may be necessary and which are positioned in the correct orientation in order to allow for protein expression. Other suitable vectors would be apparent to persons skilled in the art. By way of further example in this regard we refer to Sambrook et al. (1989).

Preferably, a polynucleotide for use in the invention in a vector is operably linked to a control sequence that is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be for example, plasmid, virus or phage vectors provided with a origin of replication, optionally a promoter for the expression of the said polynucleotide and optionally a regulator of the promoter. The vector may be one adapted to be used in vivo.

Promoters and other expression regulation signals may be selected to be compatible with the host cell for which expression is designed. Mammalian promoters, such as β-actin promoters, may be used. Tissue-specific promoters are especially preferred. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, adenovirus, HSV promoters (such as the HSV IE promoters), or HPV promoters, particularly the HPV upstream regulatory region (URR). Viral promoters are readily available in the art.

The vector may further include sequences flanking the polynucleotide giving rise to polynucleotides which comprise sequences homologous to eukaryotic genomic sequences, preferably mammalian genomic sequences. This will allow the introduction of the polynucleotides of the invention into the genome of eukaryotic cells by homologous recombination. In particular, a plasmid vector comprising the expression cassette flanked by viral sequences can be used to prepare a viral vector suitable for delivering the polynucleotides of the invention to a mammalian cell. Other examples of suitable viral vectors include herpes simplex viral vectors and retroviruses, including lentiviruses, adenoviruses, adeno-associated viruses and HPV viruses. Gene transfer techniques using these viruses are known to those skilled in the art. Retrovirus vectors for example may be used to stably integrate the polynucleotide giving rise to the polynucleotide into the host genome. Replication-defective adenovirus vectors by contrast remain episomal and therefore allow transient expression.

### Diseases and Conditions

The invention may be used to treat or prevent any suitable disease or condition, particularly one that comprises, consists essentially of, or consists of, an inflammatory condition.

In one instance, the disease or condition may be one mediated, in whole or part, by, or in which is present, one or more of the following:

(i) an antibody or antibodies;

(ii) a lymphocyte reaction;

(iii) an innate immune system reaction and/or

(iv) an alloimmune reaction.

In some cases, at least one of (i) to (iv) may be responsible for inflammation and/or damage seen in a condition. In one preferred instance, the condition or disease may be one where antibodies are responsible for damage, such as, for instance, tissue damage.

In one preferred instance, the disease may be a disease or condition mediated by an antibody or antibodies, particularly by an IgG antibody. It is well known in the art that antibodies are involved in the pathogenesis of a number of different diseases and conditions. The role of pathogenic antibodies in such diseases can be inhibited using an antibody or antibody fragment with modified glycosylation, even where the antibody or fragment recognises a different epitope to the disease mediating antibody.

For instance, examples of disease include autoimmune disorders, allergies, cardiovascular disease, cancer, infectious disease, and metabolic disease. In some instances, the condition may be where the subject has received a transplant, for instance to reduce or eliminate side effects of drug treatment given after transplantation or to reduce an immune reaction against the transplanted organ. The condition may, in some instances, be post-inflammatory side effects due to surgery.

The disease or condition can be an autoimmune disease. Such diseases include Addison's disease, alopecia areata, ankylosing spondilitis, antiphospholipid syndrome, aplastic anaemia, autoimmune gastritis, autoimmune hearing loss, autoimmune haemolytic anaemias, autoimmune hepatitis, autoimmune hypoparathyroidism, autoimmune hypophysitis, autoimmune inner ear disease, autoimmune lymphoproliferative syndrome, autoimmune myocarditis, autoimmune oophoritis, autoimmune orchitis, autoimmune polyendocrinopathy, Beçhet's disease, bullous pemphigoid, cardiomyopathy, chronic inflammatory demyelinating polyneuropathy, Churg-Strauss syndrome, coeliac disease, Crohn's disease, CREST syndrome, Degos disease, epidermolysis bullosa acquisita, essential mixed cryoglobulinaemia, giant cells arteritis, glomerulonephritis, Goodpasture's syndrome, Graves' disease, Guillan-Barre syndrome, Hashimoto's thyroiditis, idiopathic thrombocytopenic purpura, inflammatory bowel disease, Kawasaki's disease, Meniere's syndrome, mixed connective tissue disease, Mooren's ulcer, multiple sclerosis, myasthenia gravis, pemphigus foliaceous, pemphigus vulgaris, pernicious anaemia, polyarteritis nodosa, polyglandular autoimmune syndrome type 1 (PAS-1), polyglandular autoimmune syndrome type 2 (PAS-2), polyglandular autoimmune syndrome type 3 (PAS-3), polymyositis/dermatomyositis, primary biliary cirrhosis, psoriasis, psoriatic arthritis, Raynaud's syndrome, Reiter's syndrome, rheumatoid arthritis, sarcoidosis, scleroderma, Sjögren's syndrome, subacute thyroiditis, sympathetic opthalmia, systemic lupus erythematosus, Takayasu's arteritis, type 1 diabetes mellitus, vitiligo, Vogt-Koyanagi-Harada disease or Wegener's granulomatosis. Preferably the autoimmune disease is rheumatoid arthritis (RA), systemic lupus erythematosus or idiopathic thrombocytopenic purpura.

In one embodiment, the disease to be treated is an autoimmune disease and in particular one involving red blood cells, preferred examples of which include autoimmune hemolytic anaemia (AIHA), hemolytic diseases of the newborn, and Systemic Lupus Erythematosus. The disease or condition can be asthma. The asthma can be acute or chronic asthma.

In one preferred instance, the disease or condition is an immunological placenta transfer condition, particularly one selected from haemolytic disease of the fetus and newborn (HDFN) and neonatal alloimmune thrombocytopenia (NAITP).

In one instance, disease or condition may involve unwanted activation of the classical pathway of the complement system. The antibody or antibody fragment can therefore be used to treat diseases and conditions where complement activation is detrimental to the patient. For example, they can be used to treat transplantation-derived disorders, for example transplant rejection (such as acute or chronic allograft or xenograft rejection) and graft-versus-host disease. The transplantation-derived disorder may occur due to the transplantation of a tissue or an organ in a patient.

The antibody or antibody fragment are also of use in post-operative treatment, for example in the treatment of patients who have undergone heart by-pass operations. Further, the antibody or fragment can be used for the treatment of acquired haemophilia, i.e haemophiliac patients who have developed autoantibodies against coagulation factors.

In one preferred instance, the disease to be treated is selected from the group consisting of rheumatoid arthritis, immune thrombocytopenic purpura (ITP), Systemic Lupus Erythematosus (SLE), myasthenia goodpasteures and Sjögrens syndrome. In a particularly preferred embodiment, the disease to be treated is arthritis, particularly rheumatoid arthritis.

The subject is typically a mammalian subject, such as a mouse, rat or primate (e.g. a marmoset or monkey). The subject may be human or a non-human animal. The subject may be an agricultural or sports animal, for example a bovine, porcine or ovine animal, such as a cow, pig or sheep, or a horse. The animal may be a pet, such as a dog or cat.

Where the subject is a laboratory animal such as a mouse, rat or primate, the animal may be treated to induce a disease or condition mediated by pathogenic antibodies. For example, the mouse anti-CII antibody induced arthritis (CAIA) model described by Nandakumar et al. (Am. J. Pathol. 163(5): 1827-1837, 2003), or a modified version of that model, may be used.

The subject may, in one instance, be one who has not been administered a enzyme capable of modifying antibody glycosylation, for instance any of those mentioned herein and in particular EndoS. The treatment to be administered preferably does not involve administration also of an endoglycosylase. In one preferred instance, the subject has not been administered EndoS and EndoS is not administered as part of the therapy. In one instance, the invention in general may be employed where the method is not an ex vivo method and in particular where the antibody or fragment administered to the subject do not originate from the subject themselves.

The invention may be employed in relation to any disease or condition mentioned herein in relation to suppressing an immune condition, eliminating or reducing an undesired immune system effect associated with an antibody, or increasing potency of such a therapeutic antibody.

### Therapy and Prophylaxis

The present invention provides the use of an antibody or antibody fragment with modified glycosylation to treat or prevent a disease or condition comprising an inflammatory condition. In one instance, one mediated by pathogenic antibodies, particularly IgG. Treatment may be therapeutic or prophylactic. The antibodies or fragments may take advantage of the effect of modification of glycosylation to also increase the efficacy of therapeutic antibodies. Thus, invention may also be used to increase the potency of therapeutic antibodies and/or to prevent unwanted side-effects of therapeutic antibodies and to eliminate unwanted immune system effects triggered by antibody or fragment when administered as a therapeutic agent.

The antibody or fragment may be administered to an individual in order to prevent the onset of one or more symptoms of the disease or condition. In this embodiment, the subject may be asymptomatic. The subject may have a genetic predisposition to the disease. A prophylactically effective amount of the antibody or fragment is administered to such an individual. A prophylactically effective amount is an amount which prevents the onset of one or more symptoms of a disease or condition.

A therapeutically effective amount of the antibody or fragment is an amount effective to ameliorate one or more symptoms of a disease or condition. Preferably, the individual to be treated is human.

The antibody or fragment may be administered to the subject by any suitable means. The antibody or fragment may be administered by enteral or parenteral routes such as via oral, buccal, anal, pulmonary, intravenous, intra-arterial, intramuscular, intraperitoneal, intraarticular, topical or other appropriate administration routes. In one particularly preferred instance, the administration of the antibody or fragment of the invention is intravenous or subcutaneous.

The antibody or fragment may be administered to the subject in such a way as to target therapy to a particular site. For example, an antibody or fragment may be administered directly to the site of a transplanted organ. The antibody or fragment may be injected locally, for example intra-articularly or in one or more joints, particularly a joint or joints affected by arthritis. Local administration of antibody or fragment to the joints is particularly preferable for the prophylaxis or treatment of rheumatoid arthritis (RA). The antibody or fragment polypeptide may be conjugated with reagents that bind cartilage specifically.

The formulation of any of the antibody or fragment mentioned herein will depend upon factors such as the nature of the antibody or fragment and the condition to be treated. The antibody or fragment may be administered in a variety of dosage forms. It may be administered orally (e.g. as tablets, troches, lozenges, aqueous or oily suspensions, dispersible powders or granules), parenterally, subcutaneously, intravenously, intramuscularly, intrasternally, transdermally or by infusion techniques. The antibody or fragment may also be administered as suppositories. A physician will be able to determine the required route of administration for each particular patient.

Typically the antibody or fragment is formulated for use with a pharmaceutically acceptable carrier or diluent and this may be carried out using routine methods in the pharmaceutical art. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous or infusions may contain as carrier, for example, sterile water or preferably they may be in the form of sterile, aqueous, isotonic saline solutions.

For suppositories, traditional binders and carriers may include, for example, polyalkylene glycols or triglycerides; such suppositories may be formed from mixtures containing the active ingredient in the range of 0.5% to 10%, preferably 1% to 2%.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the pharmaceutical composition is lyophilised, the lyophilised material may be reconstituted prior to administration, e.g. a suspension. Reconstitution is preferably effected in buffer.

Capsules, tablets and pills for oral administration to a patient may be provided with an enteric coating comprising, for example, Eudragit “S”, Eudragit “L”, cellulose acetate, cellulose acetate phthalate or hydroxypropylmethyl cellulose.

Pharmaceutical compositions suitable for delivery by needleless injection, for example, transdermally, may also be used.

A therapeutically effective amount of an antibody or fragment is administered. The dose may be determined according to various parameters, especially according to the polypeptide or polynucleotide used; the age, weight and condition of the patient to be treated; the route of administration; and the required regimen. Again, a physician will be able to determine the required route of administration and dosage for any particular patient. A typical daily dose is from about 0.1 to 50 mg per kg, preferably from about 0.1 mg/kg to 10 mg/kg of body weight, according to the activity of the specific inhibitor, the age, weight and conditions of the subject to be treated, the type and severity of the disease and the frequency and route of administration. Preferably, daily dosage levels are from 5 mg to 2 g.

In some instances, more than one antibody or fragment with modified glycosylation may be administered to the subject. For instance, five, four, three or two different monoclonal antibodies or fragments with modified glycosylation may be administered. In some cases, five or less, preferably four or less and more preferably three or less different monoclonal antibodies or fragments with modified glycosylation may be administered.

In one preferred instance, the treatment will involve administration of the antibody or fragment direct to the subject and preferably the treatment will not involve removal of antibodies from the subject and modification of glycosylation of them before readministration. Thus, the treatment preferably lacks an ex vivo step. The treatment preferably does not involve returning EndoS treated blood to the subject. In another preferred instance, what is administered will not be a polyclonal population of antibodies. In another instance, what is administered may be polyclonal and for instance may be WIG. In a further embodiment, what is administered is an antibody or fragment which does not originate from the subject.

The subject may display, in some instances, reduced complement activation, phagocytosis, opsonisation (particularly by a therapeutic antibody), inflammatory mediator release (such as cytokine release, particularly interleukin release, especially IL-8 release), respiratory burst activation and/or degranulation. The subject may display reduced fever, swelling or other symptoms resulting from an inflammatory condition. Treatment may result in improved mobility. The subject may display reduced transplant rejection. In the instance of the administration of a therapeutic antibody, preferably the modification of full glycosylation will not prevent the therapeutic effect, thus at least some, preferably the majority and particularly preferably all of the therapeutic effect will be retained in comparison to the glycosylated form of the antibody, whilst the unlimited side effects will be reduced or eliminated. In a particularly preferred instance, hemolysis will be reduced.

Parameters which may be modulated using the invention may, in some instances, include antibody-dependent cellular cytotoxicity (ADCC), antibody-dependent cell-mediated phagocytosis (ADCP), complement-dependent cytotoxicity (CDC), CIq binding, FcR binding (half-life), protein A binding, and/or protein G binding.

The present invention is further illustrated by the following Examples which should not be construed as further limiting. The contents of all figures and all references, patents and published patent applications cited throughout this application are expressly incorporated herein by reference.

The following Examples illustrate the invention:

### EXAMPLES

### Example 1

### Monoclonal Antibodies with Modified Glycosylation Dominantly Suppress the Induction of Arthritis

Collagen antigen induced arthritis (CAIA) was used to assess the effect of antibodies with modified glycosylation on arthritis. Groups of six months old male QB=(BALB/c×B10.Q) F1 mice were injected intravenously with 9 mg of the two arthritogenic mAbs cocktail (M2=M2139 and C1=CIIC1) with or without EndoS treated (modified glycosylation) mAbs (M2D=M2139 modified glycosylation and C1D=CIIC1 with modified glycosylation) at different concentrations on day 0.

In particular, the following concentrations of monoclonal antibodies were administered:
(i) M2 (4.5 mg)+C1 (4.5 mg);
(ii) M2D (4.5 mg)+C1D (4.5 mg);
(iii) M2 (3.375 mg)+C1 (3.375 mg)+M2D (1.125 mg)+C1D (1.125 mg); and
(iii) M2 (3.9375 mg)+C1 (3.9375 mg)+M2D (0.5625 mg)+C1D (0.5625 mg).

On day 5, all the mice received 25 μg of lipopolysaccharide (LPS) from E. coli 055:B5 administered intraperitoneally. Mice were monitored for arthritis development.

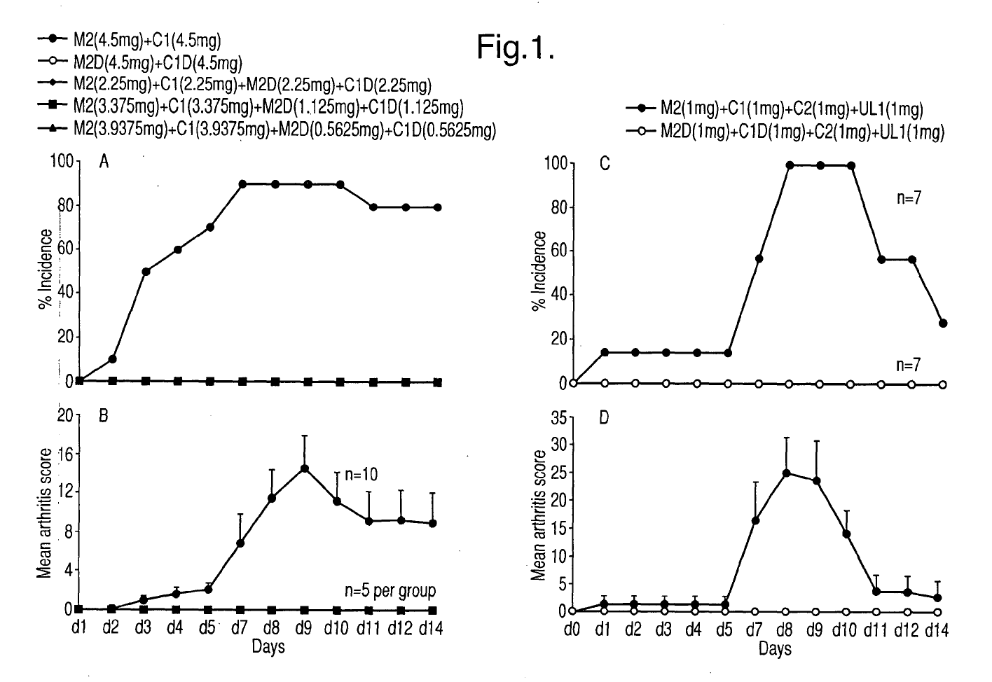

FIG. 1, shows arthritis incidence (A) and mean arthritis score (B) of arthritic animals only are shown. As can be seen, arthritis was only induced when the two monoclonal antibodies were administered in normally glycosylated form. The modified glycosylated forms of the antibodies were incapable of inducing arthritis, themselves.

Unexpectedly, not only were the modified glycosylated forms incapable of inducing arthritis, they acted dominantly, suppressing the induction of arthritis by the glycosylated forms of the antibodies at all concentrations tested, even where the disease causing glycosylated forms were present at far higher concentrations than the modified glycosylated forms.

### Example 2

### Joint Histology of CAIA mice

Joint histopathology was performed on the ankle joints of (BALB/c×B10.Q) F1 mice (n=3-4) injected with 9 mg of (i) untreated M2 and C1 monoclonal antibodies; (ii) modified glycosylation forms of the two antibodies generated using EndoS; and (iii) an equal mixture of both untreated and EndoS modified antibodies.

Ten-fold magnification showed a massive infiltration of immune cells, fibrin deposition, cartilage and bone-erosions in the mice of (i), but only very mild bone erosion in modified glycosylated mAbs treated mice. Thus, again, not only did the modified antibodies fail to induce arthritis, but they were also capable of the dominant suppression of arthritis by the glycosylated monoclonal antibodies.

### Example 3

### Antibodies with Modified Glycosylation are Capable of Dominantly Suppressing the Induction of Arthritis by Monoclonal Antibodies Against Different Epitopes

Groups of two months old male QB=(BALB/c×B10.Q)F1 mice were injected intravenously with 4 mg of the four arthritogenic mAbs cocktail (M2=M2139; C1=CIIC1; C2=CIIC2 and UL1) with or without EndoS treated (modified glycosylation) mAbs (M2D=M2139 modified glycosylation and C1D=CIIC1 modified glycosylation) on day 0. On day 5, all the mice received 25 μg per mouse of lipopolysaccharide (LPS) from E. coli 055:B5 intraperitoneally. Mice were monitored for arthritis development.

FIG. 1 shows arthritis incidence (panel C) and mean arthritis score (panel D) of arthritic animals only, with n indicating the number of mice used in each group. Error bars indicate ±SEM. As can be seen from the results depicted, even though the antibodies with modified glycosylation against M2 and C1 recognise different epitopes to the glycosylated monoclonal antibodies against C2 and UL1, they were still unexpectedly able to dominantly suppress the induction of arthritis in the mice.

### Example 4

### Inhibition of Autoantibody Destruction of Red Blood Cells

The effect of modification of glycosylation of anti-D antibodies was studied as a model to demonstrate the effect of modifying the glycosylation of antibodies already used therapeutically

### Material and Methods

### Protein and Reagents

Blood was collected from healthy donors. Plasma from patients with available clinical and laboratory information for these cases was obtained from the Haematology Department and Blood Centre at the University Hospital in Lund. Approval was obtained from the institutional review board for these studies. Full-length EndoS with glutathione-S-transferase (GST) as a fusion was recombinantly expressed and purified from Escherichia coli harboring the plasmid pGEXndoS (Collin et al (2001) Infect Immun., 69:7187-7189). RPMI 1640 medium and Hank's balanced salt solution (HBSS) were from GIBCO, Paisley, U.K. All other reagents were purchased from Sigma-Aldrich, Sweden AB, unless indicated otherwise.

### RBC Preparation and Sensitization

Blood, drawn from healthy Rh-positive individuals was collected in heparin containing tubes. After centrifugation at 1100×g for 10 minutes, plasma and buffy coat-aspirated and red blood cells were washed 5 times with 10 volumes of phosphate buffered saline (PBS): 10 mM phosphate buffer, pH 7.4, 120 mM NaCl, 3 mM KCl. RBC, 1 ml of 5% suspension, were incubated with 100 μl commercial human anti-D antibody (Rhesonativ™, Octapharma™, Lachen™, Schweiz™ or Diamed™, BIO-RAD, Hercules, Calif., USA) at 37° C. for 1 hour. RBC were then washed five times and subjected to phagocytosis assay. Sensitized RBC were assayed by ELISA using peroxidase-conjugated rabbit-anti human IgG or goat-anti rabbit IgG (BIO-RAD).

### SDS-PAGE and Western Blot Analysis

Sodium dodecyl sulfate-polyacrylamide gel electrophoresis (SDS-PAGE) was performed as previously described using Mini Protean II cell equipment from BIORAD (Hercules, Calif., USA) and the buffer system described by Laemmli. (Laemmli et al (1970) Nature, 227: 680-685). For immunoblotting, the gels were transferred to polyvinylidenefluoride (PVDF) membranes (Immobilon P, Millipore, Bedford, Mass.) as described by Matsudaira (Matsudaira et al (1987) J Biol Chem., 262: 10035-10038). After blotting, membranes were blocked in PBS supplemented with 0.05% v/v Tween 20 (PBST) and 5% w/v skim milk (DIFCO™, Detroit, Mich.) for 20 minutes at room temperature.

For detection of IgG, the blots were subsequently washed in PBST and then incubated with peroxidase-conjugated anti-human IgG or HRP anti-rabbit IgG (diluted 1:2000) (BIO-RAD, Hercules, Calif.) for one hour at 37° C. The membranes were developed using SuperSignal West Pico (PIERCE, Rockford, Ill.) according to the manufacturer's instructions before analyzing by the Chemidoc XRS imaging system and Quantity One image analysis software (BIO-RAD).

### Phagocytosis Assay

RBC sensitized with anti-D, 100-500 μl of 5% solution were added to 2-10×10⁶ blood monocytes or THP-1 cells in PBS and incubated 60 min at 37° C. in suspension with gentle agitation. Nonadherent RBC were removed by five washes with PBS. Noningested RBC were lysed with 0.5 ml distilled water for 20 second and followed with two washes with PBS. This treatment has been described to entirely lyse adherent RBC without influencing the integrity of monocytes (Bussolino et al (1987) Br J Haematol., 66:271-274).

### Quantification of RBC-Bound IgG

RBC were sensitized with human anti-D or rabbit anti-human RBC (see above) with following washes in PBS. After that cells were pelleted by centrifugation at 1000×g for five minutes, resuspended in lysis buffer containing 20 mM Tris-HCl pH 7.4, 0.150 M NaCl, 1% v/v Triton-100 and 0.25% v/v NP40 for ten minutes at 4° C. Next, the samples were centrifuged for ten minutes at 14000×g and supernatants analyzed for IgG by polyacrylamide gel/immunoblotting and ELISA.

### FITC-Labelling of RBC

Erythrocyte-labelling with fluorescein isothiocyanate (FITC) was performed according to the earlier description (Tippett et al (2007) J Immunol Methods., 325:42-50). Briefly, RBC were resuspended to 1×10⁸/ml in fresh 0.5 M sodium bicarbonate, pH 9.0 and labelled with, 200 μg/ml-500 μg/ml (FITC) (PIERCE™, Rockford, Ill.) for 30 minutes at room temperature with constant agitation following 5 washes in cold PBS. After that FITC-labelled RBC were sensitized with anti-D as described above.

### Monocyte Monolayer Assay

The MMA was performed as previously described (Nance et al (1987) Transfusion, 27:449-452). Mononuclear cells were separated from heparinised human whole blood from healthy volunteers using the Polymorphprep preparation kit (AXIS-SHIELD™, Oslo, Norway) or Ficoll-Paque Plus (Amersham Biosciences, Uppsala, Sweden) according to instructions provided by the manufacturers. After isolation, the mononuclear cells were washed 3 times with PBS and finally resuspended in RPMI-medium containg 10% fetal calf serum to the concentration 4 to 6×10⁶ cells/ml. Monocyte monolayers were prepared by incubating 0.2 ml of mononuclear cells in Lab-Tek Chamber Slides (Nunc, A/S, Roskilde, Denmark) at 37° C. for 1 hour. Nonadherent cells and RPMI were removed by aspiration with a pipette. Sensitized RBC, 0.2 ml or control RBC resuspended in RPMI containing 10% fetal calf serum were then added to the monolayer and incubated for 1 h at 37° C.

The slides were then rinsed gently with PBS to remove nonadherent red cells. Dried slides were stained with May Grünvald-Giemsa. Monocytes were examinated microscopically, and the frequency of cells with adherent or phagcytosed red cells was estimated.

### Quantification of Haemoglobin

Haemoglobin quantification was performed spectrophotometrically at 541 nm or by using the chromogen 2,7 diaminofluorene at 630 nm. Briefly, a working solution was prepared by adding 1 volume of 1% w/v 2,7 diaminofluorene in 90% glacial acetic acid to 10 volumes of 0.2 M Tris-HCl, pH 7.2 and 0.02 volumes of 30% hydrogen peroxide. 100 microliters of working solution was added to 10-100 μl of RBC supernatants. After 20 mM of incubation at room temperature, the optical density at 600 nm was measured in Wallac 1420 Multilabel Counter (PerkinElmer, Turku, Finland).

### Cell Preparations

The human monocytic cell line THP-1 (ATCC, Rockville, Md.) was cultured in RPMI 1640 medium supplemented with 10% fetal calf serum and 10 μg/ml gentamycin at 37° C. in an atmosphere containing 5% CO₂ and 95% humidity. Nunclon flasks for cell culture were used (Nunc). Monocytes were isolated from human whole blood using the Polymorphprep preparation kit (AXIS-SHIELD™, Oslo, Norway) or Ficoll-Paque Plus™ (Amersham Biosciences, Uppsala, Sweden) according to instructions provided by the manufacturers. After isolation, the cells were counted and resuspended in PBS or RPMI-medium with 10% calf serum.

### Enzyme Linked Immunosorbent Assay (ELISA)

For glycan detection, microtiter plates (NUNC, Roskilde, Denmark) were coated with 100 μl of solubilised RBC diluted 10 times in PBS and kept at 4° C. overnight. The plates were washed three times with lectin buffer containing 10 mM HEPES, pH 7.5, 0.15 M NaCl, 0.01 mM MnCl₂, 0.1 mM CaCl₂ and 0.1% v/v Tween 20 and blocked in the same buffer for one hour at room temperature.

In the next step 1 μg/ml biotinylated LCA-lectin was added and incubation continued for 1 hour at 37° C. After three washes with lectin buffer, 1 μg/ml biotinylated Lens culinaris agglutinin (LCA)-lectin (Vector Laboratories, Burlingame, Calif., USA) was added and incubation continued for 1 hour at 37° C. Following three more washes, 0.1 μg/ml peroxidase labeled streptavidin (Vector Laboratories) was added and the plate was incubated for 1 hour at 37° C.

The colour reaction was developed using tetramethylbenzidine/hydrogen peroxide containing substrate solution (R&D Systems Europe, Ltd. UK). The absorbance was read on a model Wallac 1420 Multilabel Counter (PerkinElmer, Turku, Finland) at 450 nm.

For detection of RBC-bound IgG the plate was coated with solubilized RBC diluted 10 times in PBS for 20 hours at 4° C. Next day, the plate was blocked with PBS supplemented with 0.05% v/v Tween 20 (PBST) and 2% w/v bovine serum albumin for 2 hours at room temperature. After this step, a peroxidase-antihuman IgG or peroxidase-conjugated antirabbit IgG (dilution 1:1000) (BIO-RAD, Hercules, Calif., USA) was used for detection. The plate was washed three times with PBST after the coating step and between each of the following incubation steps. The colour reaction was performed as above.

### IL-8 Assay

Human IL-8 was detected using DuoSet™ ELISA R&D Systems, Inc. MN, USA. Experiments were performed using 1×10⁶ cells per ml in 12-well cell culture plates (NUNC™, Roskilde, Denmark). Briefly, THP-1 cells or monocytes purified from peripheral blood were incubated with 0.5-1% suspension of anti-D sensitized RBC or control RBC (unsensitized) for two hours (blood monocytes) and 24 hours (THP-1 cells) at 37° C. in humidified air. The supernatants were analyzed for IL-8. Relative means and standard errors indicated with error bars were calculated from three experiments.

### C1q Uptake Studies

C1q binding to sensitized RBC were analyzed as described previously (Basta et al (1991) Blood, 78:700-702). Briefly, blood was collected into EDTA-tube. RBC 3.3×108 cells/ml in isotonic Veronal buffered saline containing 0.1% gelatin, 0.15 mM CaCl₂ and 1 mM MgCl₂ (GVBS2+). Three hundred microliters RBC were incubated with equal volume of rabbit IgG antihuman RBC, with or without treatment with EndoS, or buffer for 30 minutes at 37° C. After two washes in GVBS2+RBC pellets were resuspended in 250 μl human serum and placed in 30° C. for 15 minutes. The cell pellets were then washed and flourescein-conjugated anti-human C1q (Cappel, MP Biomedicals, LLC Solon, Ohio) added for 60 minutes incubation. After three washes of cell pellets fluorescence intensity was measured using a Wallac 1420 Multilabel Counter.

### Chemiluminescence Test (CLT)

Purified peripheral blood monocytes were washed four times in PBS containing 0.2% w/v bovine serum albumin and finally resuspended to 2×10⁶ cells/ml in Hanks balanced salt solution (HMS) containing 30% RPMI medium and 3% fetal calf serum (v/v). The cells were pipetted into 96 well microplate and incubated for 2 hours at 37° C. in 5% CO₂. RBC, 50 μl of 5% suspension in PBS, 0.5% human serum albumin (were sensitized with 50 μl plasma from anti-D positive donors on 96 V-well plate for 1 hour at 37° C. After repeated washes of plate with PBS, sensitized RBC were incubated with EndoS 10-20 μg EndoS for 30 min-1 h at 37° C. resupended in HBSS. In some cases plasma was pre-treated with EndoS before sensitization of RBC. Luminol, 0.8 mM, and RBC, 20 μl were then added to wells containing monocytes.

Chemiluminescence responses were monitored for 1 hour with 5 minutes intervals using a luminometer Wallac 1420 Multilabel Counter. Monocytes responses to sensitized RBC were compared to unsensitized control RBC and expressed as a ratio (opsonic index).

### Statistics

Student t-test were performed to determine statistical significance designated as follows: *, P<0.05; **, P<0.01; and ***, P<0.001.

### Results

The results obtained demonstrate that the use of antibodies with modified glycosylation is able to change the interaction of the antibody with the immune system, increasing potency and reducing side-effects.

### EndoS Inhibits Phagocytosis of Sensitized Red Blood Cells by Monocytes

The effects of modification of glycosylation by EndoS on IgG/Fc gamma receptor interactions was measured using autoantibody-mediated lysis of RBC, including phagocytosis via Fc gamma receptors and hemolysis, as an experimental model. For this purpose RBC were incubated with human IgG antibodies against Rhesus D antigen (anti-D), with or without preincubation of antibody with EndoS. The amount of cell-bound IgG was determined by Western immunoblot and ELISA subsequent to solubilization of sensitized RBC and IgG purification.

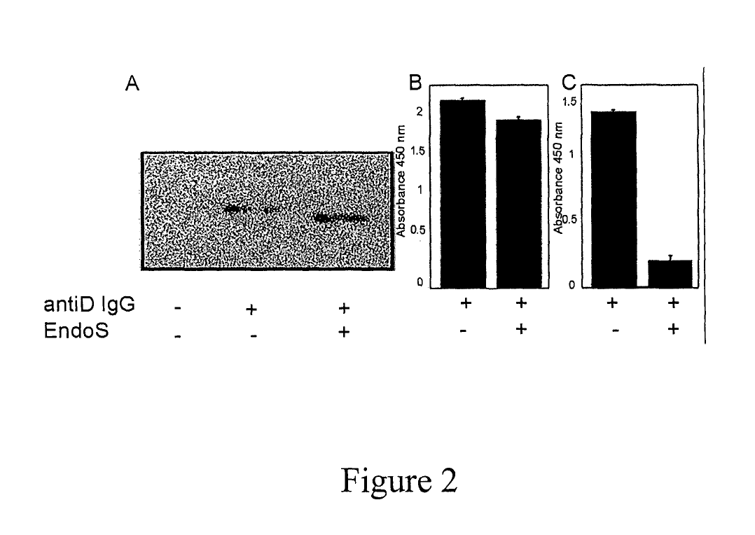

The results obtained show that the treatment of anti-D with EndoS does not affect the antigen recognition by this antibody and the amount of RBC-bound IgG was not influenced by EndoS treatment (see FIGS. 2A and 2B). Thus, modification of glycosylation does not prevent the anti-D being therapeutically effective.

EndoS hydrolysis of the N-glycan on IgG was confirmed by Western immunoblot showing a characteristic reduction in the size of IgG and decreased reactivity of EndoS-treated anti-D with Lens culinaris agglutinin (LCA) lectin using ELISA (FIGS. 2A and 2C).

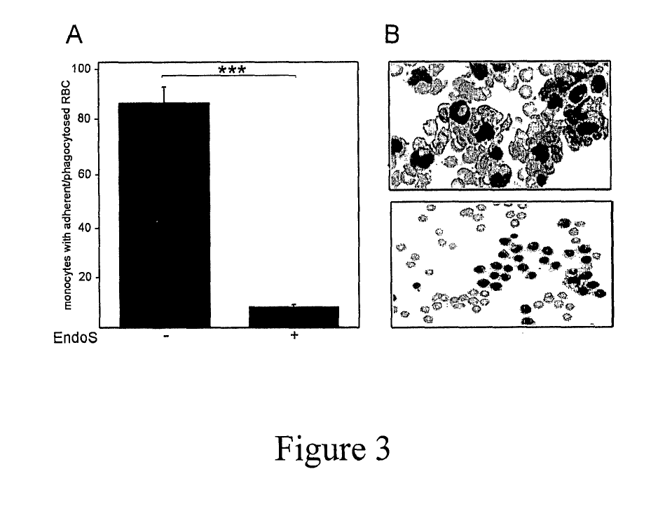

The effect(s) of modification of glycosylation on phagocytosis of anti-D IgG sensitized RBC by monocytes was analyzed using peripheral blood monocyte monolayer assay (MMA). Monolayers of monocytes purified from blood were cultured on slides and anti-D sensitized RBC were added to the monolayers and incubated. The degree of phagocytosis was determined using microscopy and expressed as amount of monocytes with adherent/phagocytosed RBC by analyzing 100 monocytes. As expected the phagocytosis of IgG-sensitized RBC was significantly greater than non-sensitized RBC and was efficiently eliminated by pre-treatment of anti-D with EndoS to achieve modification of glycosylation (FIG. 3A). Generally, the number of “rosettes” (three or more RBC attached to each monocyte) was increased for anti-D sensitized RBC but not for RBC sensitized with anti-D pre-treated with EndoS (FIG. 3B).

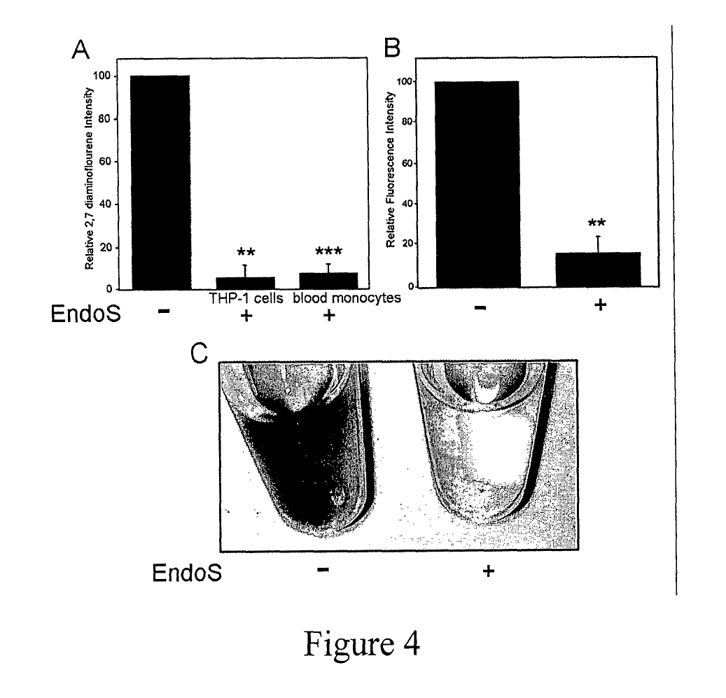

To establish the extent of the intracellular ingestion of sensitized RBC, with and without treatment of anti-D with EndoS to bring about modification of glycosylation, RBC were incubated with blood monocytes or THP-1 cells, grown in suspension. Extracellularly bound RBC were lysed with distilled water (a method that does not influence the intracellular environment of monocytes) and the amount of ingested RBC determined by measuring the concentration of internalized hemoglobin using 2,7-diaminoflourene (DAF), a reagent that is specifically oxidized by haemoglobin. This experiment showed a huge increase in accumulation of intracellular haemoglobin, explained by a phagocytosis of opsonized RBC, contrary to efficiently abolished phagocytosis of RBC sensitized with EndoS pre-treated anti-D (FIGS. 4A and 4C).

These results were confirmed using flourescein isothiocyanate (FITC)-labelled sensitized RBC and purified blood monocytes. In this experiment, erythrocytes were labelled with FITC, sensitized with anti-D, with and without treatment with EndoS. After washing the cells with PBS and removal of uningested RBC by hypotonic buffer, the degree of ingested RBC was determined by measuring the fluorescence. This experiment showed a significant decrease in phagocytosis of RBC sensitized with EndoS pre-treated anti-D (FIG. 4B). This clearly shows that the pre-treatment of anti-D with EndoS before sensitisation of RBC abrogates the phagocytosis enhancing activity of anti-D and decreases the accumulation of intracellular hemoglobin in monocytes. Thus, whilst not preventing therapeutic efficacy, modifying glycosylation does reduce or eliminate unwanted side-effects.

### EndoS Inhibits Hemolysis of Sensitized Red Blood Cells

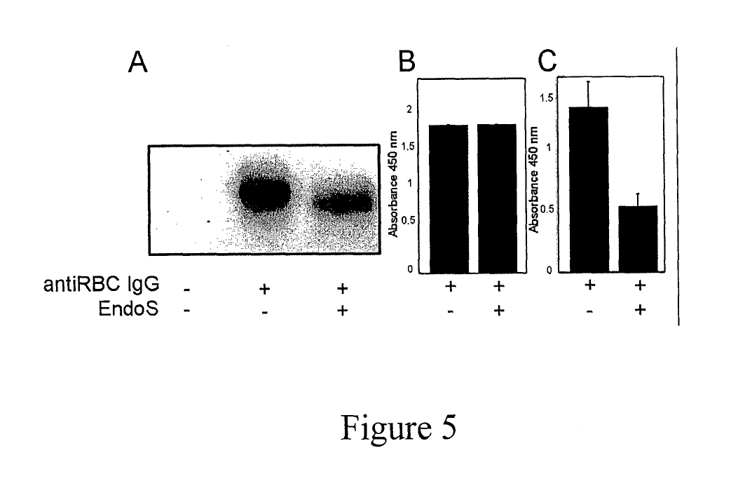

Anti-D treatment does not activate complement pathway and RBC coated with this antibody are rather cleared by mononuclear phagocytic cells via Fcgamma receptors. Therefore, in attempt to study hemolytic effects or complement activation of sensitized RBC, polyreactive rabbit anti-human RBC IgG were used. Also in this case the amount of IgG on sensitized RBC and proper hydrolysis of IgG glycan by EndoS was confirmed by Western blot and ELISA (FIG. 5A, 5B, 5C). Thus, the rabbit anti-human RBC, with and without treatment with modification of glycoslation, was added to whole blood.

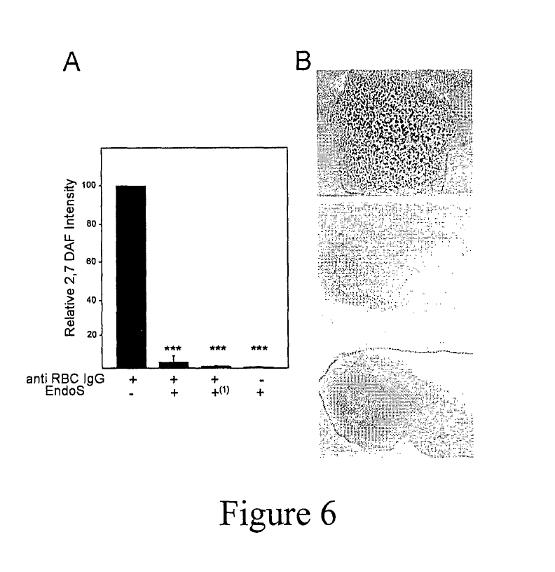

After pelleting the blood cells, the supernatants were analyzed for haemoglobin using DAF. The results present a clear increase in haemoglobin concentration using sensitized RBC. In contrast, only negligible DAF activity was seen using EndoS-treated anti-RBC, indicating inhibition of hemolysis (FIG. 6A).

It was next examined whether a simultaneous addition of EndoS and antibody to blood, without previous treatment of antibody with EndoS, could have the similar anti-hemolytic effect. It was clearly shown that pre-treatment of antibody by EndoS is not a prerequisite for inhibition of hemolysis and that a direct addition of EndoS to blood was sufficient to exclude hemolytic effect of anti-RBC (FIG. 6A). EndoS by itself did not have any effect on haemolysis (FIG. 6A). Furthermore, addition of increasing concentration of rabbit anti-human RBC IgG to whole blood caused a severe agglutination of RBC and leukocytes. Remarkably, addition of EndoS-treated antibody abrogated this effect (FIG. 6B). In conclusion, modification of glycosylation achieves anti-haemolytic activities as demonstrated by pre-treatment of anti-RBC with enzyme.

### EndoS Effectively Prevents Binding of C1q to IgG-Sensitized RBC

Human RBC were sensitized with rabbit anti-human RBC IgG, with or without pre-treatment of antibody with EndoS. Pellets of sensitized RBC were then incubated with human serum for 15 minutes at 30° C. followed by detection of complement factor C1q uptake using FITC labelled anti-human C1q.

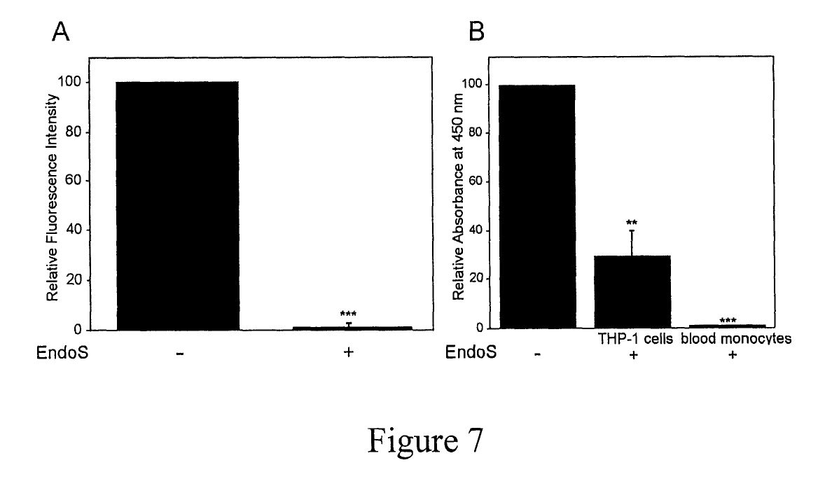

There was a significant decreased fluorescence intensity in the sample containing RBC sensitized with EndoS-treated anti-RBC, compared to RBC treated with native antibody, that indicated the inability of C1q to bind to these RBC (FIG. 7A). These results provide evidence for complement inhibitory/anti-hemolytic properties of EndoS.

### EndoS Inhibits IL-8 Secretion by Monocytes Induced by Sensitized RBC

In vitro cytokine production reveals the in vivo activity of sensitized RBC/autoantibodies predicting the extent of hemolysis and harmfulness of autoantibodies. It has been reported that interleukin-8 (IL-8) besides being produced by monocytes in response to lipopolysaccharide, tumor necrosis factor-alpha or IL-1 also has been implicated as an indicator of red blood cell incompability and mediator of the pathological events in hemolysis as for example in haemolytic transfusion reactions (Davenport et al (1994) Transfusion, 34:297-303, Davenport et al (1990) Blood, 76:2439-2442 and Wikman et al (2005) Ann Hematol., 84:150-158).

Experiments were performed to establish the role of EndoS regarding the in vitro response of monocytes exposed to sensitized RBC by measuring the release of IL-8. Thus, monocytes were incubated with anti-D sensitized RBC and the secretion of IL-8 was measured using ELISA. A significantly elevated IL-8 concentration was observed in both THP-1 cells and peripheral blood monocytes. Noticeably, RBC sensitized with EndoS-treated anti-D showed significantly decreased secretion of IL-8 in THP-1 cells and completely abolished IL-8 secretion in blood monocytes (FIG. 7B).

### EndoS Inhibits Oxygen Metabolite Production by Monocytes Due to Exposure to Sensitized RBC

To further explore the effects of EndoS on monocyte-driven cell cytotoxicity to sensitized RBC the release of reactive oxygen species using a chemiluminescent test (CLT) was measured. This method allows differentiation of the clinically significant antibodies from clinically benign and is used to predict the clinical outcome of autoantibody in transfusion of incompatible blood or severity of hemolytic disease of the newborn (Hadley et al (1999) Pediatr Nephrol., 17:91-96).

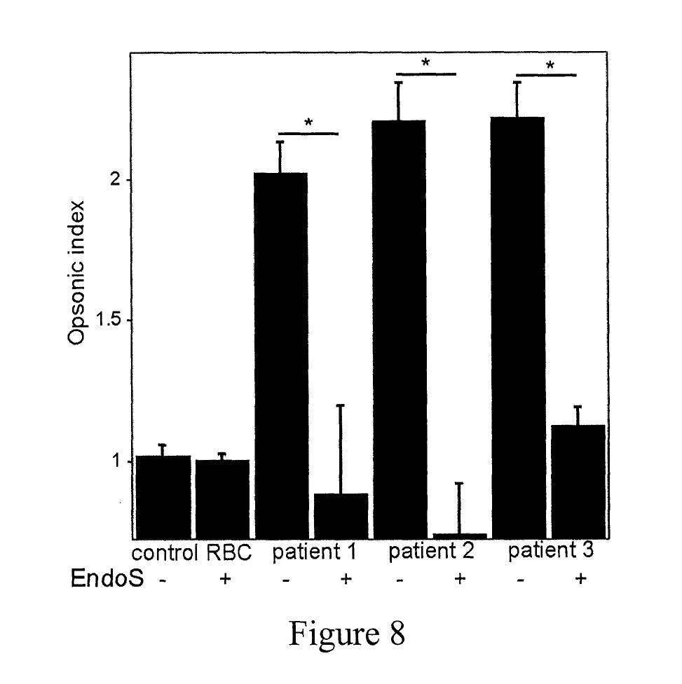

RBC were opsonised with immune plasma from three patients with positive direct antiglobulin tests (DATs) antigen. Monocytes responses to sensitized red cells were compared with their response to unsensitized cells and expressed as a ratio (opsonic index). RBC incubated with immune plasma from these patients gave positive results in CLT with opsonic index =/>2.5. In contrast, monocyte responses to RBC incubated with immune plasma pre-treated with EndoS were efficiently decreased and in some cases even below the control plasma (FIG. 8). These results indicate that treatment of immune plasma with EndoS completely abrogates in vitro cytotoxic activity of monocytes.

### Discussion

The results obtained shows how undesirable side-effects of therapeutic antibodies can be abrogated and particular negative effects of autoantibodies decreased. They demonstrated that modification of glycosylation changes the way the antibody intereacts with the immune system. Using anti-D antibodies as a model the destructive processes of opsonized RBC was eliminated by modification of glycosylation via EndoS. The results clearly demonstrate that anti-D with modified glycosylation counteracts Fc gamma receptor mediated phagocytosis of anti-D coated RBC, inhibits binding of C1q component of complement to IgG-coated RBC and minimizes cytotoxic effects of IgG anti-RBC autoantibodies, as well as oxygen radicals and secretion of IL-8 cytokine. This indicates that modification of glycosylation possesses the ability to protect RBC from lysis, predominantly extravascularly, but also intravascularly in the blood vessels, whilst not preventing the antibody acting therapeutically.

The action of anti-D or intravenous immune globulin (WIG) in ITP is thought to involve extravascular hemolysis of anti-D sensitized RBC by macrophages in the spleen which causes decreased sequestration of autoantibody sensitized platelets. However, anti-D or IVIG administrated for treatment of immune thrombocytopenic purpura (ITP) may cause severe complications such as acute hemolysis, disseminated intravascular coagulation (Gaines et al (2005) Blood, 106:1532-1537 and Gaines et al (2000) Blood 95:2523-2529) and acute renal failure (Kees-Folts et al (2002) Pediatr Nephrol., 17:91-96).

Here we show that the treatment of anti-D with EndoS to modify glycosylation, before injection, extends the survival of Ig-G-coated RBC in the circulation providing a simple, effective approach. Thus, negative side-effects of therapeutic antibodies can be reduced through modification of glycosylation.

One benefit of EndoS action is the specific activity against antibodies, without affecting RBC in binding to the cell surface or interfering with group antigens or membrane proteins (data not shown). Other endoglycosidases as EndoF from Elisabethkingiameningoseptica or EndoE from Enteroccocus faecalis have activity on a broad range of glycoproteins and may be employed.

The results presented here show the invention provides an effective approach to improve therapeutic antibodies and the way such antibodies interact with the immune system. The specific case of anti-D provides an effective model, it shows abroad biological significance not only regarding autoimmune haemolytic anemia, but also other pathological conditions characterized by an abnormal removal of antibody opsonized RBC by macrophages.

### Example 5

### Monoclonal Antibodies with Modified Glycosylation Dominantly Suppress the Induction of Arthritis by a Cocktail of Two Arthritogenic mAbs (M2139/CIIC1)

Groups of 4 months old male QB=(BALB/c×B10.Q)F1 mice were injected intravenously with a mixture either consisting of:

i) M2139D (2 mg)+CIIC1D (2 mg); or

ii) UL1D (2 mg)+CIIC2D (2 mg)

After 15 minutes the mice were injected (i.v.) with 9 mg of the cocktail of two arthritogenic anti-CII mAbs (M2139, 4.5 mg+CIIC1, 4.5 mg). On day 5, all the mice received 25 μg/i.p./mouse of lipopolysaccharide (LPS) from E. coli 055:B5. Mice were monitored for arthritis development on indicated days.

Thus, mice receiving treatment (i) were treated with EndoS-treated antibodies recognising epitopes which overlap with the epitopes recognised by the arthritogenic antibodies. Mice receiving treatment (ii) were treated with EndoS-treated antibodies recognising epitopes which do not overlap with the epitopes recognised by the arthritogenic antibodies.

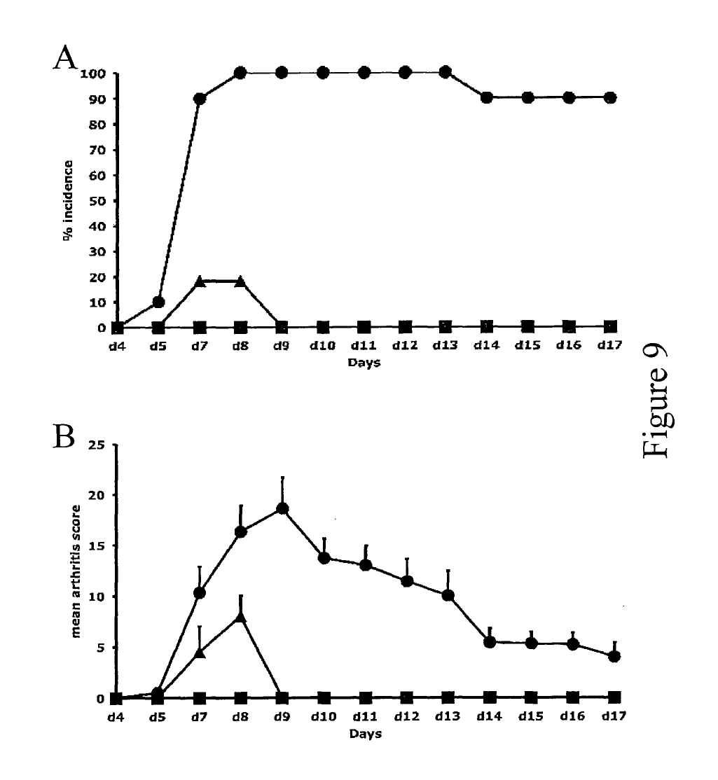

FIG. 9 shows arthritis incidence in panel A. Mean arthritis score, i.e. disease severity is shown in panel B for arthritic animals only. n indicates the number of mice used in each group. Error bars indicate ±SEM. As can be seen from the results depicted, using EndoS treated antibodies to overlapping or non-overlapping epitopes, suppresses arthritis in this model of rheumatoid arthritis. Thus, EndoS treated antibodies can dominantly suppress CAIA, i.e. suppression is separate from direct competition for epitopes.

### Example 6

### Arthritis Suppression by Antibodies with Modified Glycosylation is Independent of the Spleen

Groups of 3 months old male QB=(BALB/c×B10.Q)F1 mice were either spleenectomized or sham operated. After one month, mice were either injected intravenously with a mixture consisting of the EndoS treated antibodies M2139D (2 mg)+CIIC1D (2 mg), or left untreated. Subsequently they were injected with 9 mg of the cocktail of two arthritogenic anti-CII mAbs (M2139, 4.5 mg+CIIC1, 4.5 mg)=day 0. 25 μg/i.p./mouse of lipopolysaccharide (LPS) from E. coli 055:B5 was injected to all of the mice to enhance arthritis incidence and severity.

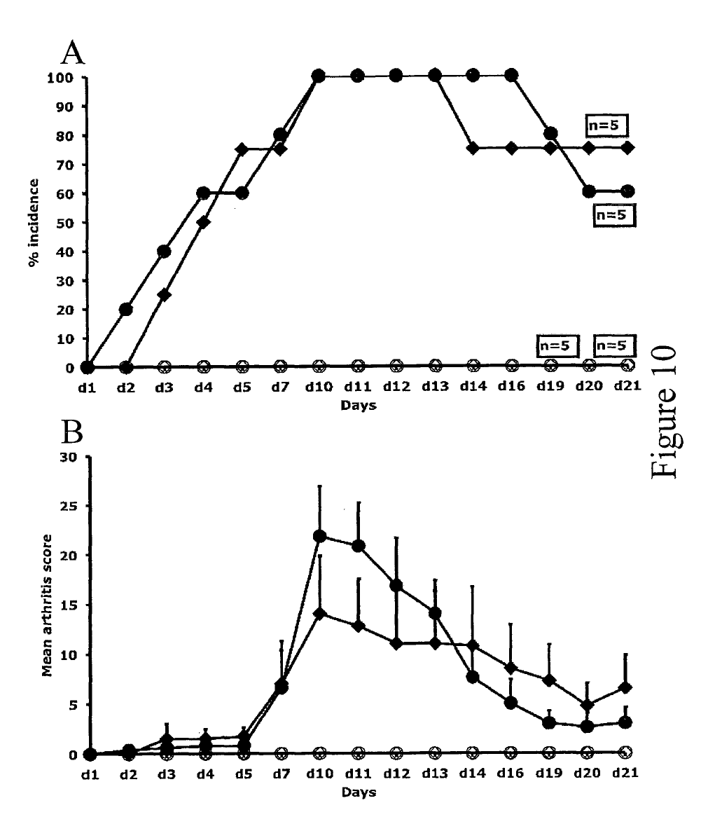

FIG. 10 shows arthritis incidence in panel A. Mean arthritis score, i.e. disease severity is shown in panel B for arthritic animals only. n indicates the number of mice used in each group. Error bars indicate ±SEM. As can be seen from the results depicted, animals which were not treated with EndoS treated antibodies experienced disease at equivalent levels of incidence and severity regardless of the presence or absence of a spleen. By contrast, using EndoS treated antibodies suppresses arthritis in this model regardless of the presence or absence of spleen. Thus, the suppression of arthritis by these antibodies is spleen-independent.

### Example 7

### Arthritis Suppression by Antibodies with Modified Glycosylation is Independent of the Antigen Specificity

Groups of 4 months old male QB=(BALB/c×B10.Q)F1 mice were either injected intravenously with a mixture consisting of the EndoS treated antibodies Hy2.15D (2 mg)+L234D (2 mg), or left untreated. Subsequently they were injected with 9 mg of the cocktail of two arthritogenic anti-CII mAbs (M2139, 4.5 mg+CIIC1, 4.5 mg)=day 0. 25 μg/i.p./mouse of lipopolysaccharide (LPS) from E. coli 055:B5 was injected to all of the mice to enhance arthritis incidence and severity. Hy2.15D is a monoclonal mouse IgG1, reactive with TNP. L234D is a monoclonal mouse IgG2a, reactive with HLA-DR.

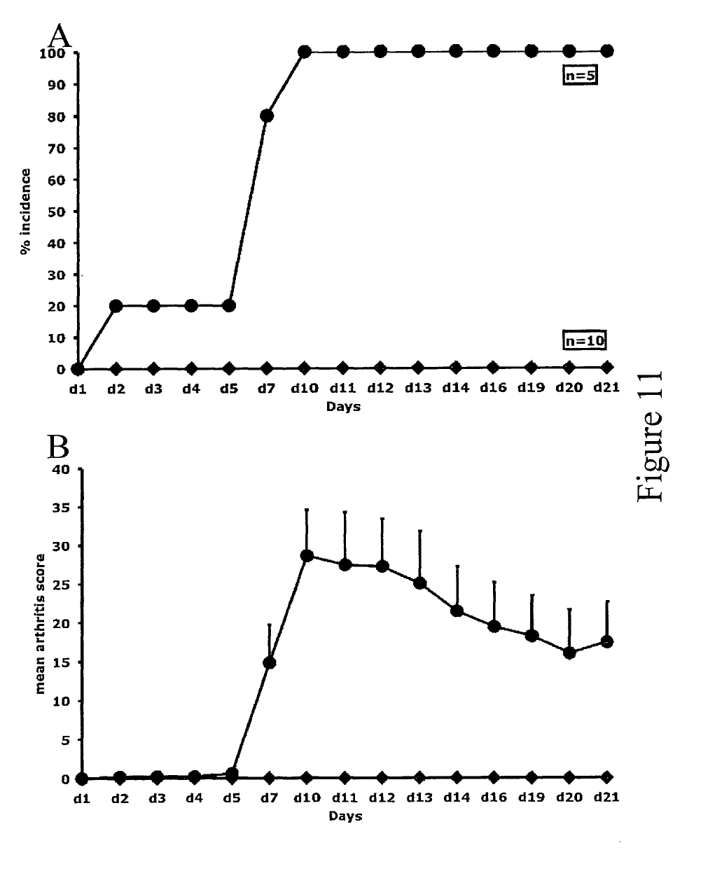

FIG. 11 shows arthritis incidence in panel A. Mean arthritis score, i.e. disease severity is shown in panel B for arthritic animals only. n indicates the number of mice used in each group. Error bars indicate ±SEM. As can be seen from the results depicted, arthritis incidence and severity is suppressed using EndoS treated antibodies which are specific for completely different antigen targets to the disease-inducing antibodies. Thus, EndoS treated antibodies can dominantly suppress CAIA, i.e. suppression is separate independent of antigen specificity.

### Example 8

### A Single Antibody with Modified Glycosylation Suppresses Arthritis at Low Dose

Groups of 4 months old male QB=(BALB/c×B10.Q)F1 mice were either injected intravenously with different concentrations (50, 250, 1000 or 4000 μg) of a single EndoS treated Ab (M2139D), or left untreated. Subsequently they were injected with 9 mg of the cocktail of two arthritogenic anti-CII mAbs (M2139, 4.5 mg+CIIC1, 4.5 mg)=day 0. 25 μg/i.p./mouse of lipopolysaccharide (LPS) from E. coli 055:B5 was injected to all of the mice to enhance arthritis incidence and severity.

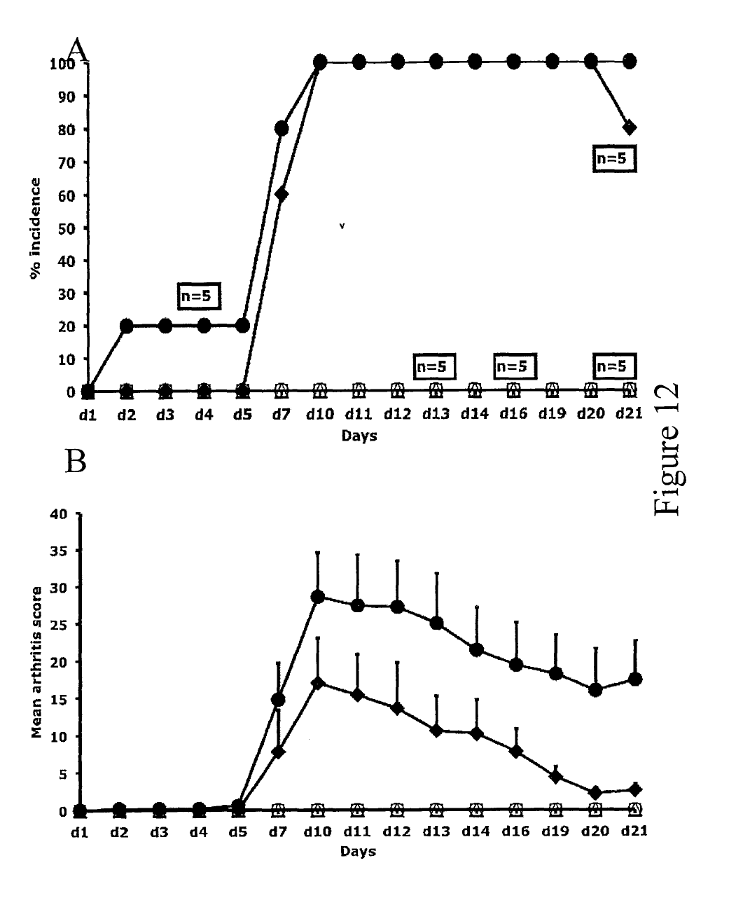

FIG. 12 shows arthritis incidence in panel A. Mean arthritis score, i.e. disease severity is shown in panel B for arthritic animals only. n indicates the number of mice used in each group. Error bars indicate ±SEM. As can be seen from the results depicted, arthritis incidence and severity is suppressed using a single EndoS treated antibody at a dose as low as 250 μg.

1.-20. (canceled)

21. An antibody, or antibody fragment, with modified glycosylation for use in treating or preventing a disease or condition, where the antibody or fragment:
(a) suppresses an inflammatory condition forming part of the disease or condition to be treated or prevented; and/or
(b) displays increased efficacy and/or decreased side effects in comparison to treatment or prevention with the corresponding antibody with unmodified glycosylation.

22. An antibody or antibody fragment according to claim 21, where the antibody or fragment is:
(i) a monoclonal antibody or monoclonal antibody fragment; or
(ii) a polyclonal antibody or polyclonal antibody fragment.

23. An antibody or antibody fragment according to claim 21, where the disease or condition is one mediated, in whole or part, by:
(i) an antibody or antibodies;
(ii) a lymphocyte reaction;
(iii) an innate immune system reaction;
(iv) an alloimmune reaction; and/or
(v) an autoimmune reaction.

24. An antibody, or antibody fragment, according to claim 21, where the antibody, or antibody fragment, to be administered binds to a disease related antigen.

25. An antibody, or antibody fragment, according to claim 21, where the antibody or fragment:
(i) binds the same epitope to a disease mediating antibody;
(ii) binds an overlapping epitope to a disease mediating antibody;
(iii) binds a different epitope to a disease mediating antibody, but on the same antigen;
(iv) binds a different epitope to a disease mediating antibody, on a different protein; or
(v) is not capable of binding antigen.

26. An antibody, or antibody fragment, according to claim 21, where the disease or condition is an autoimmune disease, post-operative treatment, acquired haemophilia or an alloimmune reaction, preferably where the alloimmune reaction is a transplant rejection, an immunological reaction upon blood transfusion or an immunological placenta transfer condition.

27. An antibody or fragment according to claim 26, wherein:
(a) said autoimmune disease is Addison's disease, alopecia areata, ankylosing spondilitis, antiphospholipid syndrome, aplastic anaemia, autoimmune gastritis, autoimmune hearing loss, autoimmune haemolytic anaemias, autoimmune hepatitis, autoimmune hypoparathyroidism, autoimmune hypophysitis, autoimmune inner ear disease, autoimmune lymphoproliferative syndrome, autoimmune myocarditis, autoimmune oophoritis, autoimmune orchitis, autoimmune polyendocrinopathy, Beçhet's disease, bullous pemphigoid, cardiomyopathy, chronic inflammatory demyelinating polyneuropathy, Churg-Strauss syndrome, coeliac disease, Crohn's disease, CREST syndrome, Degos disease, epidermolysis bullosa acquisita, essential mixed cryoglobulinaemia, giant cells arteritis, glomerulonephritis, Goodpasture's syndrome, Graves' disease, Guillan-Barre syndrome, Hashimoto's thyroiditis, idiopathic thrombocytopenic purpura, inflammatory bowel disease, Kawasaki's disease, Meniere's syndrome, mixed connective tissue disease, Mooren's ulcer, multiple sclerosis, myasthenia gravis, pemphigus foliaceous, pemphigus vulgaris, pernicious anaemia, polyarteritis nodosa, polyglandular autoimmune syndrome type 1 (PAS-1), polyglandular autoimmune syndrome type 2 (PAS-2), polyglandular autoimmune syndrome type 3 (PAS-3), polymyositis/dermatomyositis, primary biliary cirrhosis, psoriasis, psoriatic arthritis, Raynaud's syndrome, Reiter's syndrome, rheumatoid arthritis, sarcoidosis, scleroderma, Sjögren's syndrome, subacute thyroiditis, sympathetic opthalmia, systemic lupus erythematosus, Takayasu's arteritis, type 1 diabetes mellitus, vitiligo, Vogt-Koyanagi-Harada disease or Wegener's granulomatosis; or
(b) said immunological placenta transfer condition is selected from haemolytic disease of the fetus and newborn (HDFN) and neonatal alloimmune thrombocytopenia (NAITP).

28. An antibody, or antibody fragment, according to claim 26, wherein said disease is selected from: (i) rheumatoid arthritis; (ii) systemic lupus erythematosus; and (iii) idiopathic thrombocytopenic purpura.

29. An antibody, or antibody fragment, according to claim 21 where:
(i) the antibody or fragment is anti-D; or
(ii) the antibody, or antibody fragment, is provided as intravenous immunoglobulin (IVIG).

30. An antibody or fragment according to claim 26, wherein said transplant rejection is allograft or xenograft rejection.

31. An antibody or fragment according to claim 21, which has a truncated glycosyl group linked to a position in a heavy chain of the antibody or fragment, which position in a normally glycosylated antibody comprises an amino acid linked to a complex biantennary glycosyl group comprising two core GlcNAc residues.

32. An antibody according to claim 31 wherein the antibody is an IgG antibody or fragment thereof, and the position is or corresponds to any one of positions 287 to 308, preferably position 297.

33. An antibody according to claim 31, wherein the modified glycosylation is a truncated glycosyl group consisting of:
(i) a single core GlcNAc residue, optionally linked to a fucose residue; or
(ii) a first core GlcNAc residue linked to a second core GlcNAc residue, wherein the first core GlcNAc residue is optionally linked to a fucose residue; or
(iii) a first core GlcNAc residue linked to a second core GlcNAc residue linked to a mannose residue, wherein the first core GlcNAc residue is optionally linked to a fucose residue, and/or wherein the mannose residue is optionally linked to a further, single GlcNAc residue.

34. An antibody according to claim 21, wherein the modified glycosylation is a truncated glycosyl group which is N-linked or O-linked.

35. An antibody or fragment according to claim 21, wherein the antibody or fragment has been treated using the enzyme EndoS.

36. An antibody according to claim 21, wherein the modified glycosylation is replacement of the normal glycosyl group with a monosaccharide or disaccharide, an aliphatic group or compound, an aromatic group or compound, an amino acid or amino acid derivative, a nucleotide or nucleotide derivative, a lipid or lipid derivative, any organic group, or any combination thereof

37. An antibody, or antibody fragment according to claim 21 wherein:
(i) the antibody or fragment is an IgG antibody or fragment thereof; and/or
(ii) the disease or condition is one mediated by, or contributed to, in whole or part, by an antibody and in particular an IgG antibody.

38. A polynucleotide encoding an antibody, or antibody fragment, with modified glycosylation for use in treating or preventing a disease or condition, where the encoded antibody or fragment:
(a) suppresses an inflammatory condition forming part of the disease or condition to be treated or prevented; and/or
(b) displays increased efficacy and/or decreased side effects in comparision to treatment or prevention with the corresponding antibody with unmodified glycosylation.

39. A method of treating or preventing a disease or condition in a subject in need comprising administering to the subject a therapeutically effective amount of an antibody, or antibody fragment, with modified glycosylation, where the antibody or fragment:
(a) suppresses an inflammatory condition forming part of the disease or condition to be treated or prevented; and/or
(b) displays increased efficacy and/or decreased side effects in comparision to treatment or prevention with the corresponding antibody with unmodified glycosylation.
